(19) United States
(12) Patent Application Publication
(10) Pub. No.: US 2024/0315958 A1
(43) Pub. Date: Sep. 26, 2024

(54) METHOD AND DEVICE FOR PROVIDING EFFECTIVE CONTRACEPTION

(71) Applicant: Chemo Research, S.L., Madrid (ES)

(72) Inventors: Enrico COLLI, Madrid (ES); Carlos DÍEZ FUERTES, Villaquilambre (ES)

(21) Appl. No.: 18/460,441

(22) Filed: Sep. 1, 2023

(30) Foreign Application Priority Data
Mar. 23, 2023 (EP) 23382272.5

(51) Int. Cl. A61K 9/00; A61K 31/565; A61K 31/57; A61K 47/32; A61K 47/34

(52) U.S. Cl. CPC A61K 9/0039; A61K 31/565; A61K 31/57; A61K 47/32; A61K 47/34

(57) ABSTRACT

The present invention relates to a method for providing contraception in a female subject, comprising continuous intravaginal administration of levonorgestrel of from about 60 μg/day to about 100 μg/day. The invention further relates to a method for treating endometriosis, endometriosis associated pelvic pain (EAPP) and/or dysmenorrhea comprising continuously administering about 60 μg/day to about 160 μg/day of levonorgestrel. Delivery devices, such as, intravaginal rings for putting the methods in practice are also envisaged.

### TECHNICAL FIELD

The present invention pertains to the field of women's health and more specifically to a method for providing contraception comprising administering levonorgestrel whilst at the same time providing optimum estrogen suppression. A method for treatment of endometriosis and endometriosis related diseases is also envisaged as well as a drug delivery device, such as an intravaginal ring, suitable for putting these methods into practice.

### BACKGROUND

Levonorgestrel (LNG) is a synthetic progestin that exhibits no significant estrogenic activity and is highly progestational. It inhibits follicular stimulation and ovulation and reduces the cervical mucus permeability for sperm. Levonorgestrel is used as a progestogen component in numerous contraceptive products registered all over the world. It is currently one of the most widely used progestogens in combined oral contraceptive (COC) products. Cerazet® is an effective Progestin-only Pill (POP) containing desogestrel 75 μg film-coated tablets (Merck Sharp & Dohme Limited. Cerazet® 75 μg (desogestrel). Ficha técnica (SmPC Febrero 2019). Agencia Española del Medicamento y Productos Sanitarios (AEMPS). Available from: https://cima.aemps.es/cima/dochtml/p/62285/P_62285.html). Its mechanism of action focuses on inhibition of ovulation. The POP traditionally works by thickening the cervical mucus, delaying ovum transport, and providing an endometrium hostile to implantation. Cerazet® has been shown to inhibit ovulation as effectively as a COC pill. The dosage of a POP containing desogestrel 0.075 mg is sufficiently high to inhibit ovulation in at least 98% of cycles, compared with only 72% of cycles with LNG 0.030 mg ((i) Rice C, Killick S, Hickling D, Coelingh Bennink H. Ovarian activity and vaginal bleeding patterns with a desogestrel-only preparation at three different doses. Hum Reprod 1996; 11:737-40; (ii) Rice C F, Killick S R, Dieben T, Coelingh Bennink H. A comparison of the inhibition of ovulation achieved by desogestrel 75 μg and levonorgestrel 30 μg daily. Hum Reprod 1999; 14:982-5; (iii) Duijkers I J M, Heger-Mahn D, Drouin D, Skouby S. A randomised study comparing the effect on ovarian activity of a progestogen-only pill (POP) containing desogestrel and a new POP containing drospirenone in a 24/4 regimen. Eur J Contracept Reprod Health Care 2015; 20:419-27). Probably due to consistent ovulation inhibition, which is considered the most robust mechanism of action of a POP, the contraceptive efficacy of desogestrel 0.075 mg is higher than that of LNG 0.030 mg and other low-dose POPs, and comparable to the efficacy of COCs ((i) McCann M F, Potter L S. Progestin-only oral contraception: a comprehensive review. Contraception 1994; 50(Suppl 1):59-195; (ii) Collaborative Study Group on the Desogestrel-containing Progestogen-only Pill. A double-blind study comparing the contraceptive efficacy, acceptability and safety of two progestogen-only pills containing desogestrel 75 μg/day or levonorgestrel 30 μg/day. Eur J Contracept Reprod Health Care 1998; 3:169-78). Whilst Cerazet® is a widely used product with an extensive safety database, subjects receiving this treatment have reported adverse drug reactions, such as bleeding irregularities. There is therefore a need for alternative progestin only contraceptives.

Long-term implants containing only LNG were also proven to be an effective form of contraception. For example, in multicenter clinical studies with the implant Jadelle® involving 1393 women, 8 pregnancies occurred within 5 years of the placement ((i) Sivin, I. 1984. Five-year clinical studies of Norplant implants. In: The Norplant Subdermal Contraceptive System. M. M. Shaaban (ed.). Assiut, Egypt: Assiut University, pp. 74-75; (ii) Milsom I, Korver T. Ovulation incidence with oral contraceptives: a literature review. BMJ Sexual & Reproductive Health. 2008; 34:237-246).

The inhibition of ovulation is directly related with the plasma concentration of levonorgestrel in the steady state. However, the minimum threshold level of circulating LNG required for contraceptive efficacy is still uncertain. Based on early studies of Norplant® (Wyeth, Pfizer, NY), a subdermal contraceptive implant, a plasma LNG concentration of 0.3-0.4 ng/mL is often mentioned as a threshold level below which contraceptive effectiveness declines. Several weeks after insertion of the implant, mean plasma LNG levels stabilized between 0.3 and 0.4 ng/mL and declined slowly to a mean level of 0.28 ng/mL after 5 years of use; by the eighth year, the levels were around 0.22 ng/mL (range, 0.02-0.35 ng/mL). LNG concentrations associated with the occurrence of unwanted pregnancies were thus reported to be 0.21±0.06 ng/mL.

As regards the ovulation inhibition provided by levonorgestrel implants ovulation is inhibited in over 85% of the cycles in the first year of use, when the release rate of levonorgestrel is highest (V. Brache, F. Alvarez & A. Faundes (2001) Mechanism of action of levonorgestrel contraceptive implants, Gynecological Endocrinology, 15:sup2, 14-20, DOI: 10.1080/gye.15.s2.14.20). The percentage of ovulation inhibition decreases to near 65% of the cycles in years 2 and 3, while luteal activity occurs in around 50% of the cycles in the last 2 years of use. Mean estradiol serum levels during the 5-year period of Norplant were similar to those in the control group, at around 400-500 pmol/L (109-136 μg/mL).

The levonorgestrel implant exerts its contraceptive action by causing changes in the cervical mucus, by inhibiting ovulation and by promoting ovulatory dysfunction. In case of contraceptive failure, a pregnancy occurring under levonorgestrel implant is more likely to be ectopic than pregnancies conceived with other contraceptives. Due to the high contraceptive efficacy of levonorgestrel implant the absolute rate of ectopic pregnancies in Jadelle® (new brand name of Norplant®) users is very low.

Higher-dosed LNG-POPs were tested aiming for an estrogen-free contraceptive with high contraceptive reliability and an acceptable bleeding pattern (Ingrid J. M. Duijkers, Christine Klipping, Tanja Rautenberg, Barbara S. Schug, Prithi S. Kochhar, Hermann Osterwald, Michael Oettel, Effect on ovarian activity and ovulation inhibition of different oral dosages of levonorgestrel, Contraception (2022), doi:https://doi.org/10.1016/j.contraception.2022. 01.018). Investigational dosages were Levonorgestrel 0.095, 0.115 and 0.135 mg per day administered to three groups of 30 patients for 56 days. It was concluded that Levonorgestrel 0.115 mg per day was the lowest effective dose for consistent ovulation inhibition. It is known that levonorgestrel when administered orally is completely absorbed after oral administration leading to a bioavailability of nearly 100% and is not subject to first-pass metabolism, as for example shown for a commercial COC comprising levonorgestrel and ethinyl estradiol (Seasonique®).

A multicenter, open-label, single-arm study (NCT02403401) was performed to investigate the contraceptive efficacy and safety of LNG (40 μg/day) delivered via an intravaginal ring (IVR). The release rate and dose of LNG for the IVR in this study was chosen to achieve an exposure similar to that of the approved low-dose LNG POP (NorgestonVR/MicrolutVR, 30 μg/day) and the LNG implant (Norplant IIVR/JadelleVR) after a wearing period of 2 years. However, the study was prematurely terminated after approximately one-third of the planned exposure due to the high number of pregnancies.

Therefore, although the LNG safety profile is well characterized and there exists experience using various doses, pharmaceutical formulations and delivery systems, contraceptive efficacy for LNG as monotherapy administered in a vaginal delivery system has not been shown to date.

Another problem associated with progestogen-only contraceptives is menstrual irregularity due to the lack of estrogen administration. Indeed, this is the most common reason (45%) for discontinuation of levonorgestrel implant (Meirik et al (2003) “Implantable Contraceptive for women”. Human Reproduction Update, 9(1): 49-59).

Albeit the bleeding mechanism associated to progestogen-only contraceptives is still poorly understood, it is known that bleeding pattern associated with progestin-only contraceptives depend on the degree of suppression of ovarian activity. In general, if normal ovulation occurs consistently, a woman will experience menstrual bleeds at a frequency characteristic of her normal cycle. If both ovulation and follicle development are completely suppressed, amenorrhea could result. If ovulation or follicular development (and therefore estrogen secretion sufficient to stimulate endometrial growth) occurs irregularly, bleeding will be erratic and unpredictable (Glacier (2016) Anna Glasier, Chapter 134—Contraception, Editor(s): J. Larry Jameson, Leslie J De Groot, David M. de Kretser, Linda C. Giudice, Ashley B. Grossman, Shlomo Melmed, John T. Potts, Gordon C. Weir, Endocrinology: Adult and Pediatric (Seventh Edition), W.B. Saunders, 2016, Pages 2297-2309.e2).

Thus, there is a need of progestogen-only contraceptives that provide high contraceptive efficacy by inhibition of ovulation whilst at the same time providing a good bleeding profile.

Increasing the dosage of Levonorgestrel in the dosage forms will result in consistent inhibition of ovulation, however, it is desirable to achieve a complete inhibition of ovulation to improve the contraceptive efficacy, while administering the minimum dose of the drug in order to avoid or reduce side effects.

One of the main side effects is caused by estrogen suppression due to the administration of progestogen-only preparations which can lead to the occurrence of unwanted hypoestrogenic side effects, in particular bone loss. Whilst tissues vary in their sensitivity to estradiol, a mean estradiol concentration of between 30 and 45 μg/ml is assumed to be sufficient for preventing bone loss (Barbieri (1992). “Hormone treatment of endometriosis: the estrogen threshold hypothesis”. Am J Obstet Gynecol. 1992 February; 166(2):740-5. doi: 10.1016/0002-9378(92)91706-g).

The reduction in estradiol plasma levels is also related with the improvement of other conditions, such as premenstrual syndrome, hypermenorrhoea and also the improvement of other pathologies that are considered estrogen-dependent such as uterine myomatosis and subserous endometrial polyps.

Estrogen suppression also has a direct influence on the development of endometriosis. Endometriosis is a chronic, estrogen-dependent disease characterized by the presence of endometrial tissue outside the uterus including the ovaries and other pelvic structures. These lesions cause a chronic, inflammatory reaction, which can lead to the generation of scar tissue and adhesions. Women with endometriosis frequently experience symptoms of dysmenorrhea, premenstrual pain, dyspareunia and chronic fatigue, (Schindler, AE (2011). “Dienogest in long-term treatment of endometriosis.” Int J Womens Health 3: 175-184) as well as the less common symptoms of pain at ovulation, constipation, and painful urination (Taylor, HS, Giudice, LC, Lessey, BA, Abrao, MS, Kotarski, J, Archer, DF, Diamond, MP, Surrey, E, Johnson, NP, Watts, NB, Gallagher, JC, Simon, JA, Carr, BR, Dmowski, WP, Leyland, N, Rowan, JP, Duan, WR, Ng, J, Schwefel, B, Thomas, J W, Jain, R I and Chwalisz, K (2017). “Treatment of Endometriosis-Associated Pain with Elagolix, an Oral GnRH Antagonist.” N Engl J Med 377(1): 28-40). In addition, the presence of ectopic endometrium can also cause infertility, which can be the case in up to 50% of women with endometriosis (Vercellini, P, Vigano, P, Somigliana, E and Fedele, L (2014). “Endometriosis: pathogenesis and treatment.” Nature Reviews Endocrinology 10(5): 261-275). Estradiol levels around 40-60 μg/ml have been suggested in the prior art for treating endometriosis.

Currently there is no cure for endometriosis. Women with endometriosis still require ongoing, collaborative, supportive management of their condition, as well as an understanding of the significant impact that the condition can have on their quality of life. The main aims of treatment are to alleviate pain and other symptoms, reduce endometriotic lesions, and improve the quality of life of affected individuals.

Current hormonal treatments for endometriosis associated pain focus on systemic or local estrogen suppression, inhibition of tissue proliferation and inflammation, or both. Combined oral contraceptives (COCs) are widely used as the first-line treatment for dysmenorrhea or chronic pelvic pain with or without presumed endometriosis, particularly in adolescents with endometriosis (Paolo Vercellini, M. D., Laura Buggio, M. D., Maria Pina Frattaruolo, M. D., Alessandra Borghi, M. D., Dhouha Dridi, M. D., Edgardo Somigliana, M. D. (2018) “Medical treatment of endometriosis related pain” Best Practice & Research Clinical Obstetrics & Gynaecology. 51, 68-91). Nevertheless, estrogens do have a stimulatory effect on the metabolic activity of the endometrial mucosa. Thus, COCs administration could result in an estrogen dominance, with the potential risk of lesion progression (Casper, RF (2017). “Progestin-only pills may be a better first-line treatment for endometriosis than combined estrogen-progestin contraceptive pills.” Fertil Steril 107(3): 533-536).

Progestin-only treatment is also used as the first-line therapy for pelvic pain associated with endometriosis and for suppressing the extent of endometriotic lesions. One FDA approved progestin for the treatment of endometriosis, secondary amenorrhea, and abnormal uterine bleeding is norethisterone acetate (NETA) (5 mg tablets). Whilst in principle NETA can also provide ovulation inhibition starting from a dosage of 0.35 mg/daily when given continuously over 28 days, it is not approved for contraceptive use, since the high dosage needed for the treatment of endometriosis (5-15 mg/daily) is more than 10 times higher than the dosage necessary for ovulation inhibition (0.35 mg/daily). Therapy may be held at this high dosage level for a maximum of six to nine months, or until breakthrough bleeding demands temporary termination of the treatment. Furthermore, at such high dosages NETA can produce androgenic side effects, such as acne, hirsutism, weight gain and voice changes of slight severity in some women.

Another approved progestin, Dienogest (DNG), is a synthetic progestin that is currently used for clinical treatment of endometriosis in Europe with a dose of 2 mg daily (Visanne® 2 mg tablets). DNG, being devoid of androgenic activity, is better tolerated than NETA. DNG 2 mg daily provides ovulation inhibition, but ovarian activity is not completely suppressed, thus it has not been approved as a contraceptive (Caruso et al (2019). “Randomized study on the effectiveness of nomegestrol acetate plus 170-estradiol oral contraceptive versus dienogest oral pill in women with suspected endometriosis-associated chronic pelvic pain”. BMC Womens Health. 2022 May 10; 22(1):146. doi: 10.1186/s12905-022-01737-7). Therefore, users are formally invited to adopt barrier contraception or other non-hormonal alternatives (Vercellini, P, Bracco, B, Mosconi, P, Roberto, A, Alberico, D, Dhouha, D and Somigliana, E (2016). “Norethindrone acetate or dienogest for the treatment of symptomatic endometriosis: a before and after study.” Fertil Steril 105(3): 734-743.e733) when using DNG in the treatment of endometriosis.

Currently approved treatments for endometriosis associated pain have no indication for contraception. Moreover, the concomitant use of hormonal contraceptives with some FDA or European-approved drugs for endometriosis (e.g., GnRH antagonists such as the recently approved product Elagolix or progestins as dienogest) is not permitted. The need for the use of barrier contraception may limit compliance for these products and could increase the discontinuation rate. Thus, there is a definitive need for therapies treating endometriosis associated pelvic pain (EAPP) in women seeking hormonal contraception.

### SUMMARY

In view of the foregoing, one object of present disclosure is to provide for a contraceptive with a very high ovulation inhibition efficacy whilst at the same time limiting the oestrogen suppression to an optimum level, such as levels corresponding to an early follicular phase, and thus avoiding the well-known side effects caused by contraceptives of the prior art comprising levonorgestrel as an active ingredient. It is a further object of the disclosure to provide for a contraceptive with a very high ovulation inhibition efficacy which is at the same time adequate for the treatment of endometriosis, endometriosis associated pelvic pain (EAPP) and/or dysmenorrhea and other related estrogen-dependent diseases.

Embodiments of the present invention therefore relate in one aspect to a method for providing contraception in a female subject, comprising continuously administering levonorgestrel to said subject, wherein the levonorgestrel is continuously administered in an amount ranging from about 60 μg/day to about 100 μg/day, wherein the administration route is vaginal administration.

In one embodiment the present invention relates to a method for providing contraception in a female subject, comprising continuously administering levonorgestrel to said subject, wherein the average daily amount of levonorgestrel that is continuously administered is about 75 μg/day, wherein the administration route is vaginal administration.

In another aspect the present invention relates to a method for treating endometriosis, endometriosis associated pelvic pain (EAPP) and/or dysmenorrhea in a female subject, comprising continuously administering levonorgestrel to said subject, wherein the levonorgestrel is continuously administered in an amount ranging from about 60 μg/day to about 160 μg/day, for example wherein the administration route is vaginal administration.

In one embodiment the present invention relates to a method for treating endometriosis, endometriosis associated pelvic pain (EAPP) and/or dysmenorrhea in a female subject, comprising continuously administering levonorgestrel to said subject, wherein the average daily amount of levonorgestrel administered is from about 75 μg/day to about 150 μg/day, for example wherein the administration route is vaginal administration.

In one embodiment the present invention relates to a method for treating endometriosis, endometriosis associated pelvic pain (EAPP) and/or dysmenorrhea in a female subject, comprising continuously administering levonorgestrel to said subject, wherein the average daily amount of levonorgestrel administered is of about 75 μg/day, about 125 μg/day or about 150 μg/day, for example wherein the administration route is vaginal administration.

In one embodiment the present invention relates to a method for treating endometriosis, endometriosis associated pelvic pain (EAPP) and/or dysmenorrhea as described above, wherein said treatment also provides contraception.

In one embodiment of the methods of present invention no further contraceptive ingredient is administered to the female subject at the same time, for example in one embodiment no estrogen is administered to the female subject at the same time.

In one embodiment of the methods of present invention said administering of Levonorgestrel also induces amenorrhea.

In a further aspect the present invention relates to a method for providing contraception in a female subject, comprising continuously administering Levonorgestrel at an amount from about 60 μg/day to about 200 μg/day to a female subject, wherein the administration route is vaginal administration.

In one embodiment of said aspect the present invention relates to a method for providing contraception in a female subject, comprising continuously administering Levonorgestrel at an average amount of between about 75 μg/day to about 150 μg/day to a female subject. In one embodiment, the administration route is vaginal administration.

In one embodiment of said aspect the present invention relates to a method for providing contraception in a female subject, comprising continuously administering Levonorgestrel at an average amount of about 75 μg/day, about 125 μg/day, or about 150 μg/day to a female subject. In one embodiment, the administration route is vaginal administration.

In a specific embodiment the administration route is transmucosal, such as vaginal.

In a further embodiment of the methods disclosed herein, no further contraceptive ingredient, for example in some embodiments no estrogen is administered to the female subject at the same time.

In one embodiment of the disclosed methods said administering of Levonorgestrel also induces amenorrhea.

In a further aspect the present invention relates to a drug delivery device comprising
(a) a core comprising a polymer;
(b) a sheath substantially or completely surrounding said core;
   said sheath comprising a polymer; and
(c) Levonorgestrel dissolved or dispersed in said core and/or said sheath, wherein the total amount of Levonorgestrel present in said core/or said sheath is between about 9 mg to about 11 mg.

In a specific embodiment the total amount of levonorgestrel present in said core/or said sheath is about 10 mg.

In one embodiment the polymer of the core is a polyurethane and the polymer of the sheath is an ethylene-vinylacetate copolymer.

In one embodiment the ethylene-vinylacetate copolymer of the sheath comprises a vinyl acetate content comprised from 10 to 40% w/w, for example 15 to 30% w/w.

In one further embodiment, the delivery device comprises
(a) a core comprising a polyurethane;
(b) a sheath substantially or completely surrounding said core,
   said sheath comprising ethylene vinyl acetate copolymer with a vinyl acetate content comprised from about 10 to 40% w/w, for example from about 15 to 30% w/w; and
(c) Levonorgestrel dissolved or dispersed in said core and/or said sheath, wherein the total amount of Levonorgestrel present in said core/or said sheath is about 10 mg.

In one further embodiment, the delivery device comprises
(a) a core comprising a polyurethane;
(b) a sheath substantially or completely surrounding said core,
   said sheath comprising ethylene vinyl acetate copolymer with a vinyl acetate content of about 18% w/w; and
(c) Levonorgestrel dissolved or dispersed in said core and/or said sheath, wherein the total amount of Levonorgestrel present in said core/or said sheath is about 10 mg.

In one embodiment of said drug delivery device, the device releases Levonorgestrel at a constant rate during 28 days after having been administered to the female subject.

In one embodiment the present invention relates to a drug delivery device comprising levonorgestrel wherein said device releases:
no more than about 150 μg, for example no more than about 130 μg, of levonorgestrel during an initial 24-hour period of release; and
about 60 μg to 90 μg of levonorgestrel per day for at least 27 days after the initial 24 hours period of release,
when the device is subjected to an in vitro release test in a 0.2M sodium acetate buffer with 1.0% sodium lauryl sulfate (SLS) surfactant adjusted to pH 4.2 medium.

In one embodiment the present invention relates to a drug delivery device comprising levonorgestrel wherein said device releases:
about 70 μg to about 150 μg, for example about 70 μg to about 130 μg of levonorgestrel during an initial 24-hour period of release; and
about 60 μg to 90 μg, for example about 60 μg to about 80 μg of levonorgestrel per day for at least 27 days after the initial 24 hours period of release,
when the device is subjected to an in vitro release test in a 0.2M sodium acetate buffer with 1.0% sodium lauryl sulfate (SLS) surfactant adjusted to pH 4.2 medium.

In some embodiments the average daily release of levonorgestrel over the treatment cycle of 28 days, including the initial 24-hour period of release and the subsequent 27 days after the initial 24 hours period of release, is from about 60 μg to about 100 μg, for example about 70 μg to about 80 μg, or about 75 μg per day.

In one embodiment the daily release of levonorgestrel during the treatment cycle of 28 days, including the initial 24-hour period of release and the subsequent 27 days after the initial 24 hours period of release, is on no day less than about 56 μg per day.

In a further embodiment the present invention relates to a drug delivery device comprising levonorgestrel wherein said device releases:
no more than about 250 μg of levonorgestrel during an initial 24-hour period of release; and
about 90 μg to 150 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In a further embodiment the present invention relates to a drug delivery device comprising levonorgestrel wherein said device releases:
about 100 μg to about 200 μg, for example about 110 μg to about 170 μg of levonorgestrel during an initial 24-hour period of release; and
about 90 μg to 150 μg, for example about 90 μg to 140 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In some embodiments the average daily release of levonorgestrel over the treatment cycle of 28 days, including the initial 24-hour period of release and the subsequent 27 days after the initial 24 hours period of release, is from about 90 μg to about 160 μg, for example from about 105 μg to about 140 μg, or about 125 μg per day.

In one embodiment the daily release of levonorgestrel during the treatment cycle of 28 days, including the initial 24-hour period of release and the subsequent 27 days after the initial 24 hours period of release, is on no day less than about 87 μg per day.

In a further embodiment the present invention relates to a drug delivery device comprising levonorgestrel wherein said device releases:
no more than about 300 μg of levonorgestrel during an initial 24-hour period of release; and
about 110 μg to 180 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one embodiment said device releases:
no more than about 250 μg, for example about 170 μg to about 220 μg of levonorgestrel during an initial 24-hour period of release; and
110 μg to 180 μg, for example about 120 μg to about 170 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one embodiment the average daily release of levonorgestrel over the treatment cycle of 28 days, including the initial 24-hour period of release and the subsequent 27 days after the initial 24 hours period of release, is from about 120 μg to about 200 μg, for example about 150 μg per day.

In one embodiment the daily release of levonorgestrel during the treatment cycle of 28 days, including the initial 24-hour period of release and the subsequent 27 days after the initial 24 hours period of release, is on no day less than about 105 μg per day.

In one embodiment, the release of levonorgestrel from the delivery device as described herein follows zero order kinetics after the initial 24 hours period of release, i.e., the amount of levonorgestrel released each day is constant.

In a certain embodiment the levonorgestrel is administered continuously for 28 days via the delivery device after the device has been placed in the subject.

In a specific embodiment the delivery device according to present invention does not comprise a further active ingredient.

In another specific embodiment the delivery device according to present invention does not comprise a further contraceptive ingredient.

In another yet another specific embodiment, the delivery device does not comprise estrogen.

In one embodiment of the drug delivery device of present invention levonorgestrel is present in the core at a concentration of 0.20 to 1.00 wt % based on the total core weight.

In a further embodiment the sheath has a thickness comprised from 5 to 500 μm, for example from 50 to 200 μm.

In one embodiment the drug delivery device of the present invention provides a mean Cmax value for levonorgestrel of less than 1 ng/ml after one treatment cycle of 28 days and of less than 0.7 ng/ml after two treatment cycles of 28 days each and a mean AUC (0.-t) value of less than 350 h*ng/ml after one treatment cycle of 28 days and of less than 370 h*ng/ml after two treatment cycles of 28 days each in a female subject, after the delivery device has been placed intravaginally within the subjects body.

In another embodiment the drug delivery device of the present invention provides a mean Cmax value for levonorgestrel of less than 1.6 ng/ml after one treatment cycle of 28 days and of less than 1 ng/ml after two treatment cycles of 28 days each and a mean AUC (0-t) value of less than 580 h*ng/ml after one treatment cycle of 28 days and of less than 540 h*ng/ml after two treatment cycles of 28 days each in a female subject, after the delivery device has been placed intravaginally within the subjects body.

In another embodiment the drug delivery device of the present invention provides a mean Cmax value for levonorgestrel of less than 1.5 ng/ml after one treatment cycle of 28 days and of less than 1 ng/ml after two treatment cycles of 28 days each and a mean AUC (0-t) value of less than 480 h*ng/ml after one treatment cycle of 28 days and of less than 540 h*ng/ml after two treatment cycles of 28 days each.

In one embodiment the drug delivery device of the present invention has a shape selected from a spiral (helicoidal) shape, a T-shape or a ring shape, for example in some embodiments the drug delivery device has a ring shape. In a specific embodiment the device is a vaginal ring.

### BRIEF DESCRIPTION OF THE DRAWINGS

FIG. 1: In-vitro Elution profile of Levonorgestrel Vaginal drug delivery system 75 μg/day.

FIG. 2: In-vitro Elution profile of Levonorgestrel Vaginal drug delivery system 125 μg/day.

FIG. 3: In-vitro Elution profile of Levonorgestrel Vaginal drug delivery system 150 μg/day

FIG. 4: Estradiol Level by Visit and Body Mass Index. Mean estradiol levels in each treatment cycle and by BMI category for all treatment groups are shown.

FIG. 5: Pharmacokinetic Parameters for LNG. Mean concentration-time profiles of LNG and of participants from the LNG VDS 75, 125 and 150 treatment groups.

FIG. 6: Pharmacokinetic Parameters for SHBG. Mean concentration-time profiles of LNG and SHBG, respectively, of participants from the LNG VDS 75, 125 and 150 treatment groups.

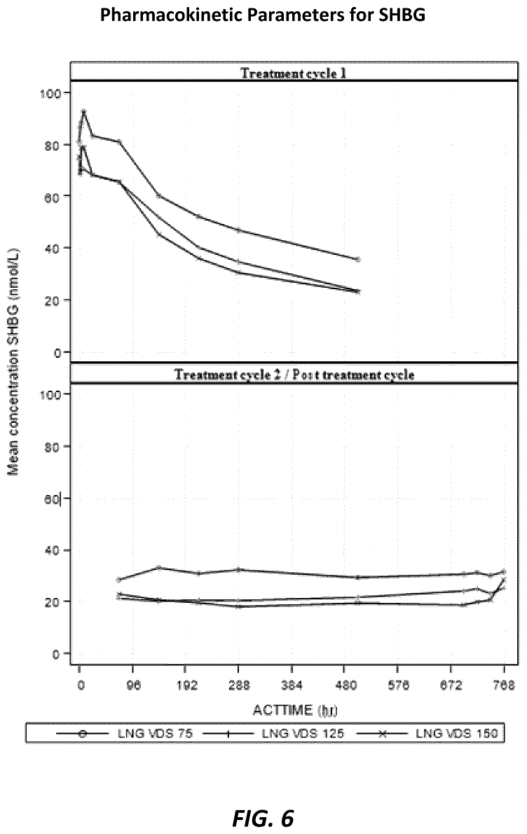

FIG. 7: Pharmacokinetic Parameters for LNG by Body Mass Index. Mean concentration-time profiles of LNG and of participants from the LNG VDS 75, 125 and 150 treatment groups categorized by BMI.

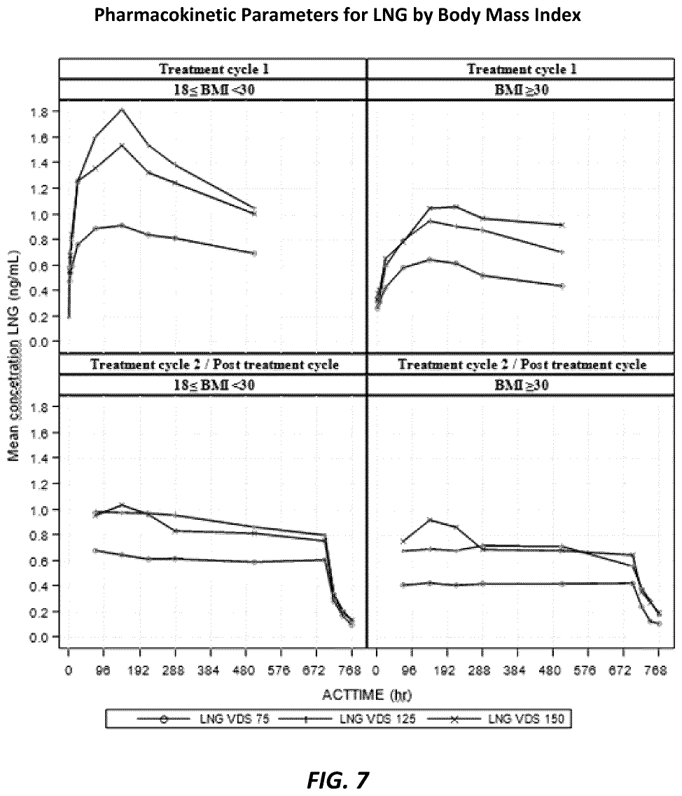

FIG. 8: Pharmacokinetic Parameters for SHBG by Body Mass Index. Mean concentration-time profiles of LNG and SHBG, respectively, of participants from the LNG VDS 75, 125 and 150 treatment groups categorized by BMI.

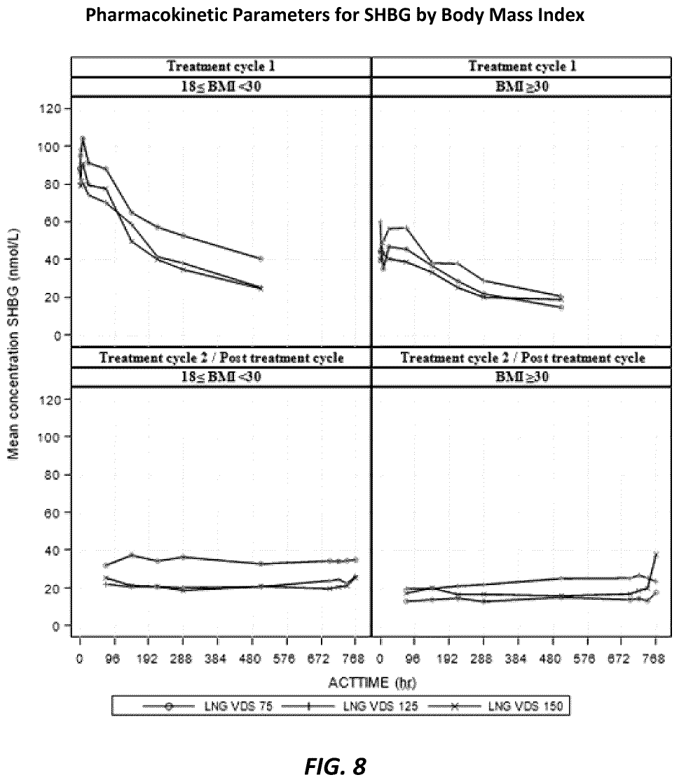

FIG. 9: Schematic depiction of three shapes of drug delivery devices A) ring shaped, B) T-shaped, C) spiral (helicoidal) shape.

### DETAILED DESCRIPTION

As described above it was an object of certain embodiments of the present invention to provide for a contraceptive with a very high ovulation inhibition efficacy whilst at the same time limiting the estrogen suppression to an optimum level, such as levels corresponding to an early follicular phase, and thus avoiding the well-known side effects caused by contraceptives of the prior art comprising levonorgestrel as an active ingredient.

The inventors have therefore set out to develop a delivery device with the desired properties and have performed a multi-center, phase 2, open-label, randomized clinical trial to evaluate inhibition of ovulation of levonorgestrel (LNG) being released in three different dosing strengths (75 μg/day, 125 μg/day and 150 μg/day) from a LNG vaginal delivery system (LNG VDS) during 28 days in continuous regimen versus orally administered desogestrel (Cerazet®) in healthy female subjects aged 18-35 years.

The study consisted of four phases, a screening phase of a minimum of 4 weeks and maximum of 8 weeks if washout cycle was needed), a pre-treatment cycle of 28 days, a treatment cycle (TC) consisting of 56 treatment days (2 cycles TC1 and TC2 each 28 days per cycle) and a post-treatment cycle of 28 days.

Ovulation inhibition was measured by assessing the ovarian activity studying follicular growth, estradiol and progesterone serum concentrations in the patients.

Furthermore, the influence of LNG VDS on the cervical mucus, the endometrial thickness, the return of ovulation in the post-treatment cycle, the impact of LNG VDS on the blood levels of sexual hormones and the safety and tolerability of LNG VDS were assessed during this trial.

Overall, 268 subjects were screened, of whom 137 were randomized and 130 started the study treatment. The efficacy was assessed in a total of 128 participants comprising the full analysis set or FAS, and in 118 participants comprising the per protocol set or PP. For the PK/PD analysis, 55 participants comprised the PK population (14 out of them had a BMI ≥30 kg/m2 and 41 had a BMI between ≥18 and <30 kg/m2).

It could be shown that a total of 127/128 (99.2%) participants showed inhibition of ovulation during TC1. The only participant who did not show inhibition of ovulation belonged to the BMI group ≥18 and <30 kg/m2 and was treated with Cerazet. During TC2, all participants surprisingly reached total inhibition of ovulation (125/125 [100%]).

The numbers and percentages of participants presenting none or minimum ovarian activity (Hoogland score 1-2) were 19 (57.6%), 17 (56.7%), 26 (76.5%) and 26 (83.9%) in the Cerazet, LNG VDS 75, 125 and 150 treatment groups, respectively, in TC1 and 15 (48.4%), 19 (63.3%), 28 (84.8%) and 27 (87.1%), in the Cerazet, LNG VDS 75, 125 and 150 treatment groups, respectively, in TC2. The numbers and percentages of participants who presented residual ovarian activity (Hoogland score 3-4) were 13 (39.4%), 13 (43.3%), 8 (23.5%), 5 (16.1%) in the Cerazet, LNG VDS 75, 125 and 150 treatment groups, respectively, in TC1 and 16 (51.6%), 11 (36.7%), 5 (15.2%) and 4 (12.9%) in the Cerazet, LNG VDS 75, 125 and 150 treatment groups, respectively, in TC2.

Only 1 (0.8%) participant had a positive result in the Landgren test during TC1. This participant was treated with Cerazet. No positive results in the Landgren score were observed in TC2 in any of the groups.

In summary, this clinical trial data convincingly shows that levonorgestrel administered via a delivery device according to present invention effectively inhibited ovulation in all three releasing rates, i.e., 75 μg/day, 125 μg/day and 150 μg/day, regardless of the BMI of the subject.

Furthermore, the safety profile of the LNG VDS was acceptable, and no serious treatment-emergent adverse events were observed during the trial.

The present invention therefore relates in one aspect to a method for providing contraception in a female subject, comprising continuously administering levonorgestrel to said subject, wherein the levonorgestrel is continuously administered in an amount ranging from about 60 μg/day to about 100 μg/day, wherein the administration route is vaginal administration.

In one embodiment the average daily amount of levonorgestrel administered is about 75 μg/day, wherein the administration route is vaginal administration.

As described above previous studies have shown that Levonorgestrel delivered at 115 μg/day was the lowest effective dose for consistent ovulation inhibition when administered orally. It is known that levonorgestrel when administered orally is completely absorbed after oral administration leading to a bioavailability of nearly 100% and is not subject to first-pass metabolism.

Surprisingly it has now been found that the very low dose of Levonorgestrel of 75 μg/day when administered vaginally completely, or substantially completely, inhibited ovulation.

It is known that the ovarian suppression is dose dependent, the higher the doses of LNG, the greater ovarian suppression and the lower estradiol levels are achieved. Surprisingly, with a lower dose of levonorgestrel such as 75 μg/day of levonorgestrel, 100% of ovulation inhibition is observed. The administration of such a low dose of levonorgestrel is highly advantageous since lower side effects are expected.

As described above, one of the main side effects is caused by estrogen suppression due to the administration of progestogen-only preparations which can lead to the occurrence of unwanted hypoestrogenic side effects, in particular bone loss. Whilst tissues vary in their sensitivity to estradiol a mean estradiol concentration of between 30 and 45 μg/ml is assumed to be sufficient for preventing bone loss.

Thus, it is essential to ensure a good balance between the estrogen suppression and the contraceptive effect because the estradiol levels must be maintained within a certain safety level to avoid the loss of the bone density undesirable side effect.

It has been found that the estrogen suppression caused by the administration of about 75 μg/day of LNG after two treatment cycles is within the desirable security range, i.e. above 30 μg/ml.

The clinical trial data has shown that the mean (SD) estradiol concentration was 97.1 (84.3) pg/mL, 59.3 (40.2) pg/mL, 37.3 (21.2) pg/mL and 32.1 (12.1) pg/mL in the Cerazet, LNG VDS 75, 125 and 150 treatment groups, respectively, in TC1, and 66.5 (41.6) pg/mL, 48.0 (21.4) pg/mL, 30.4 (10.4) pg/mL and 26.8 (8.2) pg/mL in the Cerazet, LNG VDS 75, 125 and 150 treatment groups, respectively, in TC2.

The serum estradiol levels observed for the three doses of levonorgestrel IVR after two cycles of administration tested resulted in that the lower dose of Levonorgestrel 75 μg/day of LNG after two treatment cycles leads to less estrogen suppression than the higher doses of 125 μg/day and 150 μg/day and thus, lower effect on bone loss.

In another embodiment, the reduction in estradiol plasma levels is also related to the improvement of other conditions, such as premenstrual syndrome, hypermenorrhoea and also the improvement of other pathologies that are considered estrogen-dependent such as uterine myomatosis and subserous endometrial polyps.

In particular, the estrogen suppression is directly related with the improvement of endometriosis. Estrogens play a key role in the pathophysiology of endometriosis, since they promote the implantation of endometrial tissue in the peritoneum, have proliferative and antiapoptotic effects in endometrial cells, and stimulate local and systemic inflammation (21, 22). Barbieri explained that estradiol's levels should be around 40-60 μg/ml in order to treat endometriosis.

It has been found that the estrogen suppression caused by the administration of about 75 μg/day of LNG after two treatment cycles is within the desirable range to treat endometriosis, i.e., 40-60 μg/ml as explained above.

### Definitions

As used herein the term “amenorrhea” refers to the absence/lack of bleeding/spotting during at least 56 days or two administration cycles in a female subject, for example a woman of reproductive age.

As used herein the term “burst release” refers to a rate of release over time of an active pharmaceutical ingredient wherein the rate is not uniform but is generally greater during a given period of time, typically immediately following emplacement of the device bearing active pharmaceutical ingredient in tissue.

As used herein the term “complete inhibition of ovulation” relates to 100% ovulation inhibition in a subject. The term “substantially complete inhibition of ovulation” is to be understood to be 90%, 91%, 92%, 93%, 94%, 95%, 96%, 97%, 98% or 99% ovulation inhibition in a subject.

As used herein a “contraceptive method” or “method for providing contraception” relates to a method for preventing pregnancy.

As used herein the term “dysmenorrhea” refers to the medical term for painful menstrual periods which are caused by uterine contractions. Primary dysmenorrhea refers to recurrent pain, while secondary dysmenorrhea results from reproductive system disorders.

The term “dispersed”, as used herein, means that one or more active pharmaceutical ingredients form a dispersion in the core polymer or sheath polymer, so that they are partially or completely in solid particulate form suspended in and surrounded by a continuous phase.

As used herein, the term “dissolved”, means that one or more active pharmaceutical ingredients form a solution in the core polymer or the sheath polymer, so that they are distributed in the core polymer or the sheath polymer forming a homogeneous phase.

As used herein the term “endometriosis” and “endometriosis associated pelvic pain (EAPP)” refer to a chronic, estrogen-dependent disease that is characterized by the formation of endometriotic lesions outside the uterus including the ovaries and other pelvic structures and one of its most common symptoms which is reported as pelvic pain, respectively. All subtypes of endometriosis, including superficial, cystic, deep infiltrating, abdominal wall and catametial endometriosis are included. The efficacy of the management of Endometriosis associated pelvic pain (EAPP) can be assessed using different rating scales, such as the visual analogue scale (VAS) or the numeric rating scale (NRS), as well known to the skilled person (see for example Gerlinger et al. (2010) and Breivik et al. (2008)). Depending on the rating scale for example already a difference of at least 1.0, at least 1.1, at least 1.2, at least 1.3, at least 1.4, at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3.0 on a 0-10 NRS scale to placebo can be regarded as clinically meaningful and as providing a real benefit to the patient.

As used herein, the term “estrogen(s)” defines a group of steroid hormones which promote the development and maintenance of female characteristics of the body. Synthetic estrogens are well-known and commonly used in oral contraceptives or to treat menopausal and menstrual disorders.

As used herein, the term “levonorgestrel” or “LNG” refers to levonorgestrel itself, i.e., the chemical entity identified by the CAS registry Number 797-63-7, solvates of levonorgestrel, and derivates or prodrugs of levonorgestrel.

As used herein the term “post treatment cycle” or “post treatment” refers to the 28 days after the TC starting from day 1 after removal of the LNG VDS or day 1 of no oral administration of Cerazet.

As used herein “progestogen-only contraceptive”, or “progestogen-only pill” (also known as “POP”) means a pill or a contraceptive which comprises progestogens as sole contraceptive active ingredients and does not comprise any estrogen.

As used herein a “therapeutically effective amount” refers to an amount effective, at dosages and for periods of time necessary to achieve the desired therapeutic result, such as one or more of the following therapeutic results, such as a significant delay of the onset or progression of the disease; or a significant reduction of the severity of one or more symptoms. A therapeutically effective amount is also typically one in which any toxic or detrimental effect of the active ingredient or pharmaceutical composition is outweighed by the therapeutically beneficial effects.

As used herein, the term “treatment cycle” (TC) refers to a total of 56 continuous treatment days. Treatment cycle 1 (TC1) refers to the first cycle consisting of 28 days and Treatment Cycle 2 (TC2) to the second cycle consisting of 28 days.

As used herein, “treatment”, “treating” or “treat” refer to: (i) preventing or retarding a disease, disorder or condition from occurring in a subject which may be predisposed to the disease, disorder and/or condition but has not yet been diagnosed as having it; (ii) inhibiting the disease, disorder or condition, i.e., arresting or slowing down its development or progression; and/or (iii) relieving the disease, disorder or condition, i.e., causing regression of the disease, disorder and/or condition. In certain embodiments, such term refers to the amelioration or eradication of a disease or symptoms associated with a disease.

As used herein, “vaginal administration” and “intravaginal administration” can be used interchangeably. They refer to the administration of a compound, for example levonorgestrel, via the vaginal mucosa.

As used herein, “zero order” or “near zero order” means that a substantially constant amount or a constant amount of drug per unit time is released over a given period of time. For the purposes of the invention, the term “substantially constant amount” is as defined by the Higuchi formula, see Journal Pharmaceutical Sciences 1963, vol. 52, 1145-1149.

### Methods

The present invention relates in one aspect to a method for providing contraception in a female subject, comprising continuously administering levonorgestrel to said subject, wherein the levonorgestrel is continuously administered in an amount ranging from about 60 μg/day to about 100 μg/day.

In one embodiment the present invention relates to a method for providing contraception in a female subject, comprising continuously administering levonorgestrel to said subject, wherein the average daily amount of levonorgestrel administered is about 75 μg/day, wherein the administration route is vaginal administration.

It could be surprisingly shown that the average dose of 75 μg/day when administered intravaginally, provides for complete or substantially complete inhibition of ovulation. At the same time the dose of 75 μg/day when administered intravaginally provides less estrogen suppression than the higher doses tested after two cycles of administration, thus diminishing the unwanted side effects caused by estrogen suppression, such as bone loss.

It has been also found that the estrogen suppression caused by the average administration of about 75 μg/day of LNG after two treatment cycles is within the desirable range to treat endometriosis, e.g., 40-60 μg/ml as explained above.

The present invention therefore in another aspect relates to a method for treating endometriosis, endometriosis associated pelvic pain (EAPP) and/or dysmenorrhea in a female subject, comprising continuously administering levonorgestrel to said subject, wherein the levonorgestrel is continuously administered in an amount ranging from about 60 μg/day to about 160 μg/day, for example in one embodiment the administration route is vaginal administration.

In one embodiment the present invention relates to a method for treating endometriosis, endometriosis associated pelvic pain (EAPP) and/or dysmenorrhea in a female subject, comprising continuously administering levonorgestrel to said subject, wherein the average daily amount of levonorgestrel administered is from about 75 μg/day to about 150 μg/day.

In one embodiment the present invention relates to a method for treating endometriosis, endometriosis associated pelvic pain (EAPP) and/or dysmenorrhea in a female subject, comprising continuously administering levonorgestrel to said subject, wherein the average daily amount of levonorgestrel administered is of about 75 μg/day, about 125 μg/day or about 150 μg/day, for example in one embodiment the administration route is vaginal administration.

In one embodiment, the average daily amount of levonorgestrel administered is about 75 μg/day to about 125 μg/day.

In a further embodiment, said treatment also provides contraception. As was described in more detail above the administration of all three dosages of the clinical trial provided for a complete, or substantially complete ovulation inhibition.

In one specific embodiment of the methods of present invention the administration route is transmucosal, for example vaginal administration.

In a further embodiment of the methods of present invention no further contraceptive ingredient is administered to the female subject at the same time.

In a further embodiment of the methods of present invention no estrogen is administered to the female subject at the same time.

In one embodiment of the method of present invention administering of levonorgestrel as described herein above induces amenorrhea.

The present invention in another aspect relates to a method for treating other pathologies that are considered estrogen-dependent, such as uterine myomatosis and subserous endometrial polyps, comprising administering levonorgestrel to a subject continuously as described herein.

In a further aspect the present invention relates to a method for providing contraception in a female subject, comprising continuously administering Levonorgestrel at an amount of from about 60 μg/day to about 200 μg/day to a female subject, wherein the administration route is vaginal administration.

In one embodiment of said aspect the present invention relates to a method for providing contraception in a female subject, comprising continuously administering Levonorgestrel at an average amount of between about 75 μg/day to 150 μg/day to a female subject, wherein the administration route is vaginal administration.

In one embodiment of said aspect the present invention relates to a method for providing contraception in a female subject, comprising continuously administering Levonorgestrel at an average amount of about 75 μg/day, about 125 μg/day, or about 150 μg/day to a female subject, for example in one embodiment the administration route is vaginal administration.

In a further embodiment, no further contraceptive ingredient, for example no oestrogen is administered to the female subject at the same time.

In one embodiment, administering of Levonorgestrel also induces amenorrhea.

In one other embodiment of a method comprising the use of levonorgestrel, within 28 days post-treatment ovulation is recovered in more than 80% of the female subjects. In contrast in the Cerazet treatment group only in 69.7% of the female subject's ovulation is recovered within 28 days post-treatment.

### Device

In a further aspect the present invention relates to a drug delivery device comprising
(a) a core comprising a polymer;
(b) a sheath substantially or completely surrounding said core;
   said sheath comprising a polymer; and
(c) Levonorgestrel dissolved or dispersed in said core and/or said sheath, wherein the total amount of Levonorgestrel present in said core/or said sheath is between about 9 mg to about 11 mg.

In one embodiment the total amount of levonorgestrel present in said core/or said sheath is about 10 mg.

In one embodiment of said aspect the polymer is selected from low density polyethylene, ethylene-vinylacetate copolymers, styrene-butadiene-styrene copolymers, polyurethanes, poly(dimethyl siloxane), or silicone polyether amide copolymer, silicone, silicone-poly(carbonate urethane), poly(carbonate urethane), and silicone-poly(ether urethane), or combinations thereof.

In one embodiment the polymer of the core is a polyurethane and the polymer of the sheath is an ethylene-vinylacetate copolymer.

In one embodiment the ethylene-vinylacetate copolymer of the sheath comprises a vinyl acetate content comprised from 10 to 40% w/w, for example 15 to 30% w/w.

In one embodiment the present invention relates to a drug delivery device comprising
(a) a core comprising a polyurethane;
(b) a sheath substantially or completely surrounding said core and
   said sheath comprising ethylene vinyl acetate copolymer (EVA) with a vinyl acetate content comprised from 10 to 40% w/w, for example 15 to 30% w/w;
(c) Levonorgestrel dissolved or dispersed in said core and/or said sheath, wherein the total amount of Levonorgestrel present in said core/or said sheath is about 10 mg.

In one further embodiment, the delivery device comprises
(a) a core comprising a polyurethane;
(b) a sheath substantially or completely surrounding said core; and
   said sheath comprising ethylene vinyl acetate copolymer with a vinyl acetate content of about 18% w/w;
(c) Levonorgestrel dissolved or dispersed in said core and/or said sheath, wherein the total amount of Levonorgestrel present in said core/or said sheath is about 10 mg.

The core of the device comprises a polyurethane. Polyurethane (PU) is a polymer composed of a chain of organic units joined by carbamate (urethane) links. Examples of suitable polyurethanes, which can be used as core polymers include, without limitation, aliphatic polyether-based thermoplastic polyurethanes, aliphatic hydrophilic polyether-based thermoplastic polyurethanes, aromatic polyether-based thermoplastic polyurethanes, aliphatic polycarbonate-based thermoplastic polyurethanes, aromatic polycarbonate-based thermoplastic polyurethanes, aromatic polyether based polyurethane elastomers, thermoplastic polyether poly(urethanes), thermoplastic silicone polyether polyurethanes, thermoplastic silicone polycarbonate polyurethanes and hydrophilic thermoplastic polyurethane elastomers, or combinations thereof. Commercially available suitable polyurethanes include, without limitation, Tecophilic®, Tecoflex®, Tecothane®, Carbothane®, Chronothane®, Elasthane®, Pursil®, Hydrothane® and PATHWAY®. Specific examples are the polyurethanes Hydrothane® AL25 80A and PATHWAY® PY-PT80AE25.

PATHWAY PY-PT80AE25 is an aliphatic, polyether-based thermoplastic polyurethane provided by LUBRIZOL.

Hydrothane® AL25 80A is as disclosed in U.S. Pat. No. 9,872,829 B2.

In one embodiment, the core polymer comprises at least 50% polyurethane. In one embodiment, the core comprises at least 60% polyurethane. In one embodiment, the core comprises at least 70% polyurethane. In one embodiment, the core comprises at least 80% polyurethane. In one embodiment, the core comprises at least 90% polyurethane. In one embodiment, the core comprises at least 95% polyurethane. In one embodiment, the core consists essentially of polyurethane, i.e., the core comprises from 50 to 100%, more particularly 75 to 100% of polyurethane. The above percentages refer to weight percentages (polyurethane weight in respect to core weight).

The core may further comprise one or more of the following additives: release-modifying substances including, without limitation, polyethylene glycerol, glucose, glycine, ascorbic acid, hydroxyethylcellulose, croscarmellose, lactose; fillers including, without limitation, high surface area fumed and precipitated silicas, clays such as kaolin, crushed quartz, diatomaceous earths, calcium carbonate, barium sulphate, iron oxide, titanium dioxide and carbon black; antioxidants including, without limitation, octadecyl-3-(3,5-di-tert.butyl-4-hydroxyphenyl)-propionate (Irganox®), ethylenediaminetetraacetic acid (EDTA), butylated hydroxytoluene (BHT), citric acid (CA), butylated hydroxyanisole (BHA), tertiary butylhydroquinone (TBHQ), and propyl 30 gallate (PG) and alpha-tocopherol; lubricants including, without limitation, irgawax, talc, aerosil and stearates such as magnesium stearate; and excipients including, without limitation, water-soluble or water-swellable polysaccharides, such as croscarmellose (cross linked carboxymethyl cellulose) or hydroxyethylcellulose, glucose, lactose or other mono- or di-saccharides, or their water-soluble salts, proteins such as gelatin, nonionic surface active agents, bile salts, organic solvents, such as ethoxydiglycol, polyethylene glycol and fatty acid esters, or combinations thereof.

As used herein, the expression a sheath “substantially surrounding the core” means that at least 90% of the core surface area, more particularly 95%, more particularly 100%, is surrounded by the sheath. In an embodiment, the sheath of ethylene vinyl acetate copolymer completely surrounds the core.

As mentioned above, the sheath of the device comprises ethylene vinyl acetate (EVA) copolymer. EVA is a semi-crystalline copolymer of ethylene and vinyl acetate (VA) monomers. The specific ethylene vinyl acetate copolymer of the sheath to be used will depend on the desired drug flux and can be any commercially available ethylene vinyl acetate copolymer. In one embodiment, optionally in combination with one or more features of the various embodiments described above or below, the sheath comprises an EVA copolymer having a vinyl acetate (VA) content comprised from 1 to 50% w/w, or from 10 to 40% w/w, or even 15 to 30% w/w. In a specific embodiment, the EVA sheath copolymer has a vinyl acetate content of 15 to 20% w/w, or about 18% w/w.

For the purposes of embodiments of the invention, the “vinyl acetate content” refers to the vinyl acetate content in weight based on the total weight of the ethylene vinyl acetate copolymer.

Suitable commercially available ethylene vinyl acetate copolymers include the products available under the trade names: Elvax®, VitalDose®, Evatane®, Lupolen V®, Movriton®, Ultrathene®, Ateva®, Vestypar®, Dupont 760, Equistar UE637-000, Huntsman PE1903, and F 100309 (Exxon Mobil).

In one embodiment, the sheath comprises at least 50%, at least 60%, at least 70%, at least 80%, at least 90%, at least 95% ethylene vinyl acetate. In one embodiment, the sheath consists essentially of ethylene vinyl acetate, i.e., the sheath comprises from 50 to 100%, or from 75 to 100% of ethylene vinyl acetate. The above percentages refer to weight percentages (ethylene vinyl acetate weight in respect to sheath weight).

The sheath may further comprise one or more of the following additives: a release-modifying substances including, without limitation, polyethylene glycerol, glucose, glycine, ascorbic acid, hydroxyethylcellulose, croscarmellose, lactose; fillers including, without limitation, high surface area fumed and precipitated silicas, clays such as kaolin, crushed quartz, diatomaceous earths, calcium carbonate, barium sulphate, iron oxide, titanium dioxide and carbon black; antioxidants including, without limitation, octadecyl-3-(3,5-di-tert.butyl-4-hydroxyphenyl)-propionate (Irganox®), ethylenediaminetetraacetic acid (EDTA), butylated hydroxytoluene (BHT), citric acid (CA), butylated hydroxyanisole (BHA), tertiary butylhydroquinone (TBHQ), propyl 30 gallate (PG) and alpha-tocopherol; lubricants including, without limitation, irgawax, talc, aerosil and stearates such as magnesium stearate; and excipients including, without limitation, water-soluble or water-swellable polysaccharides, such as croscarmellose (cross linked carboxymethyl cellulose) or hydroxyethylcellulose, glucose, lactose or other mono- or di-saccharides, or their water-soluble salts, proteins such as gelatin, nonionic surface active agents, bile salts, organic solvents, such as ethoxydiglycol, polyethylene glycol and fatty acid esters, or combinations thereof.

In a specific embodiment, optionally in combination with one or more features of the various embodiments described above or below, the core weight represents from 70 to 95 wt % of the total device weight, and the sheath weight represents from 5 to 30 wt % of the total device weight, being the total device weight 100%.

In another specific embodiment, the LNG is present in the core. In a more specific embodiment, LNG is present in the core in a concentration which is lower than its saturation concentration at 25° C.

The inventors have found that levonorgestrel is very soluble in the core polymer, in particular at the below mentioned concentrations. Thus, LNG is present in a concentration which is lower than its saturation concentration, and hence there is no tendency for the LNG to crystallize over time, at any practical temperature. As a consequence, the devices of the invention are stable when stored at room temperature over prolonged periods of time, in particular over at least 6 months. This has the advantage that the devices do not require expensive storage and transport below room temperature.

In one embodiment of the drug delivery device of present invention the core comprises a hydrophilic thermoplastic polyurethane, for example PATHWAY® PY-PT80AE25 and levonorgestrel at a concentration of 0.46% w/w and 0.51% w/w and a polymeric sheath of EVA with a vinyl acetate content of 18% (w/w).

The inventors have found that, when the devices of the invention, in particular the above-mentioned devices, are subjected to tissue or in-vitro release media, the levonorgestrel is eluted at or near zero order, thus minimizing potential peak/trough fluctuations and side effects, while maximizing the amount of time the drug concentrations remain within the therapeutic window (efficacy). By zero or near zero order is meant herein that a substantially constant amount or a constant amount of drug per unit time is released over a given period of time. For the purposes of the invention, the term “substantially constant amount” is as defined by the Higuchi formula, see Journal Pharmaceutical Sciences 1963, vol. 52, 1145-1149.

Additionally, the above-mentioned devices also show low initial burst release. The term “burst release” refers to a rate of release over time of an active pharmaceutical ingredient wherein the rate is not uniform but is generally greater during a given period of time, typically immediately following emplacement of the device bearing active pharmaceutical ingredient in tissue.

In one embodiment the present invention relates to drug delivery device comprising levonorgestrel wherein said device releases:
no more than about 150 μg, or no more than about 130 μg of levonorgestrel during an initial 24-hour period of release; and
about 60 μg to 90 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one embodiment the present invention relates to drug delivery device comprising levonorgestrel wherein said device releases:
about 70 μg to 150 μg, or about 70 μg to 130 μg of levonorgestrel during an initial 24-hour period of release; and
about 60 μg to 90 μg, or about 60 μg to 80 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one embodiment, the device releases
about 90 μg to 140 μg of levonorgestrel during an initial 24-hour period of release and;
about 65 μg to 80 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one aspect of the above embodiments at least 56 μg, or about 56 μg to about 90 μg of levonorgestrel are released on day 28.

In one aspect of the above embodiments the daily release of levonorgestrel during the treatment cycle of 28 days, including the initial 24-hour period of release and the subsequent 27 days after the initial 24 hours period of release, is on no less than about 56 μg per day.

In another embodiment the average daily release of levonorgestrel over the treatment cycle of 28 days, including the initial 24-hour period of release and the subsequent 27 days after the initial 24 hours period of release, is from about 60 μg to about 100 μg, or about 70 μg to about 80 μg, or about 75 μg per day.

One other embodiment the present invention relates to drug delivery device comprising levonorgestrel wherein said device releases:
no more than 250 μg of levonorgestrel during an initial 24-hour period of release; and
about 90 μg to 150 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In a further embodiment the present invention relates to a drug delivery device comprising levonorgestrel wherein said device releases:
about 100 μg to about 200 μg, or about 110 μg to about 170 μg of levonorgestrel during an initial 24-hour period of release; and
about 90 μg to 150 μg, or about 90 μg to 140 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In another embodiment the device releases:
no more than 220 μg of levonorgestrel during an initial 24-hour period of release; and
about 105 μg to 135 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one aspect of the above embodiments more than about 85 μg to 95 μg of levonorgestrel are released on day 28.

In one aspect of the above embodiments the daily release of levonorgestrel during the treatment cycle of 28 days, including the initial 24-hour period of release and the subsequent 27 days after the initial 24 hours period of release, is on no less than about 87 μg per day.

In still another embodiment the average daily release of levonorgestrel over the treatment cycle of 28 days, including the initial 24-hour period of release and the subsequent 27 days after the initial 24 hours period of release, is from about 90 μg to about 160 μg, or from about 105 μg to about 140 μg, or about 125 μg per day.

In one other embodiment the present invention relates to drug delivery device comprising levonorgestrel wherein said device releases:
no more than 300 μg of levonorgestrel during an initial 24-hour period of release; and
about 110 μg to 180 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In a different embodiment said device releases:
no more than 250 μg, or about 170 μg to about 220 μg of levonorgestrel during an initial 24-hour period of release; and
about 110 μg to 180 μg, or about 120 μg to about 170 μg of levonorgestrel per day for at least 27 days after the initial 24 hours period of release
when the device is subjected to an in vitro release test in a 0.2M sodium acetate buffer with 1.0% sodium lauryl sulfate (SLS) surfactant adjusted to pH 4.2 medium.

In one aspect of the above embodiments more than about 100 μg to 110 μg, or example more than about 105 μg of levonorgestrel are released on day 28.

In one embodiment the daily release of levonorgestrel during the treatment cycle of 28 days, including the initial 24-hour period of release and the subsequent 27 days after the initial 24 hours period of release, is on no day less than about 105 μg per day.

In more embodiments the average daily release of levonorgestrel over the treatment cycle of 28 days, including the initial 24-hour period of release and the subsequent 27 days after the initial 24 hours period of release, is about 120 μg to about 200 μg, or about 150 μg per day.

In a specific embodiment, the release of levonorgestrel from the delivery device as described herein follows zero order kinetics after the initial 24 hours period of release, i.e., the amount of levonorgestrel released each day is constant.

In one embodiment the constant release means that the amount of levonorgestrel released per day can vary up to a maximum of 20 to 25%.

In an embodiment the intravaginal ring according to present invention does not comprise a further active ingredient.

In one embodiment of the drug delivery device of present invention levonorgestrel is present in the core at a concentration of about 0.20 to 1.00 wt % based on the total core weight.

In a further embodiment the sheath has a thickness comprised from about 5 to 500 μm, or from about 50 to 200 μm.

In one embodiment the drug delivery device of present invention provides a mean Cmax value for levonorgestrel of less than 1 ng/ml after one treatment cycle of 28 days and of less than 0.7 ng/ml after two treatment cycles of 28 days each and a mean AUC (0.-t) value of less than 350 h*ng/ml after one treatment cycle of 28 days and of less than 370 h*ng/ml after two treatment cycles of 28 days each in a female subject, after the delivery device has been placed intravaginally within the subjects body.

In another embodiment the drug delivery device of present invention provides a mean Cmax value for levonorgestrel of less than 1.6 ng/ml after one treatment cycle of 28 days and of less than 1 ng/ml after two treatment cycles of 28 days each and a mean AUC (0-t) value of less than 580 h*ng/ml after one treatment cycle of 28 days and of less than 540 h*ng/ml after two treatment cycles of 28 days each in a female subject, after the delivery device has been placed intravaginally within the subjects body.

In another embodiment the drug delivery device of present invention provides a mean Cmax value for levonorgestrel of less than 1.5 ng/ml after one treatment cycle of 28 days and of less than 1 ng/ml after two treatment cycles of 28 days each and a mean AUC (0-t) value of less than 480 h*ng/ml after one treatment cycle of 28 days and of less than 540 h*ng/ml after two treatment cycles of 28 days each.

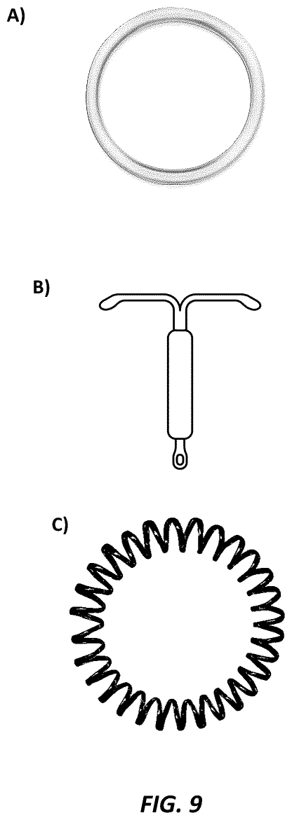

In one embodiment the drug delivery device of present invention has a shape selected from a spiral shape (helicoidal shape), a T-shape or a ring shape, for example in some embodiments the drug delivery device has a ring shape. The various shapes are depicted FIG. 9 A)-C). In a certain embodiment the device is a vaginal ring. In some embodiments, the ring-shaped device has an outer diameter comprised from 50 to 60 mm, more particularly from 52 to 56 mm, and an inner from 40 to 48 mm, or from 44 to 48 mm and has a cross sectional diameter comprised from 2.5 to 8 mm, for example 4 mm.

In some aspects, this dosage form shall not contain any estrogen which, whilst being beneficial for the contraceptive effect, can be detrimental for any estrogen induced diseases.

It is contemplated that any features described herein can optionally be combined with any of the embodiments of any medical or contraceptive use, composition, kit, contraceptive methods, methods of treatment, or method of manufacturing of the invention; and any embodiment discussed in this specification can be implemented with respect to any of these. It will be understood that particular embodiments described herein are shown by way of illustration and not as limitations of the invention.

All publications and patent applications are herein incorporated by reference in their entireties to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, or 10%. Accordingly, the term “about” may mean the indicated value ±5% of its value, for example the indicated value ±2% of its value, or in some embodiments the term “about” means exactly the indicated value (±0%).

The following examples serve to illustrate the present invention and should not be construed as limiting the scope thereof.

### EXAMPLES

### Example 1

### The Vaginal Delivery Systems (VDS) Comprise the Following Composition

TABLE 1
Formulation for the three different VDS comprising different dosage strength

| | | Quantitative Composition | | | | | |
|---|---|---|---|---|---|---|---|
| | | 150 μg/day | | 125 μg/day | | 75 μg/day | |
| Ingredients | Function | % | mg | % | mg | % | mg |
| Levonorgestrel | Active | 0.51 | 10.80 | 0.51 | 10.80 | 0.46 | 9.72 |
| | substance | | | | | | |
| Polyurethane | Carrier | 94.31 | 1994.03 | 92.59 | 1952.90 | 86.73 | 1813.41 |
| | polymer | | | | | | |
| Ethylene vinyl | Control | 5.18 | 109.60 | 6.98 | 145.40 | 12.81 | 267.75 |
| acetate 18% | Releasing | | | | | | |
| vinyl acetate | Polymer | | | | | | |

| | | | | | | |
|---|---|---|---|---|---|---|
| Total | 100.0 | 2114.4 | 100.0 | 2109.1 | 100.0 | 2090.9 |

### Manufacturing Process

The VDS were manufactured with the process comprising the following steps:
1) Polymer drying: Drying of the thermoplastic polyurethane elastomer, PY-PT80AE25, for at least 6 hours in an air dryer at 80° C.
2) Blend: GlenMills T2F turbula mixer was used to homogenously blend the materials. The following materials were added to the bin in the following order: a) Fraction A of thermoplastic polyurethane elastomer, b) micronized Levonorgestrel, c) Fraction B of thermoplastic polyurethane elastomer and blended for 30 minutes at 13 rpm.
3) Compounding: A Leistritz ZSE18 twin screw extruder was used to compound the LNG into thermoplastic polyurethane elastomer. The dry blended LNG and polymer were fed into the extruder by gravimetric feeder and extruded out through a strand die with a circular orifice. Extruder process zones set at 130-150° C. and screw speed set at 120-180 rpm. The extruded strands were cooled down in a water bath and directly fed to the pelletizing unit.
4) Stand Pelletizing: The cooled fibre was directly pulled by the pelletizing unit and pelletized into 3.0 mm length pellets.
5) Pellets drying: The API loaded polymer pellets were dried for at least 4 hours in an air dryer; at 80° C.
6) Blending: dry pellets were mixed during 5 minutes at 13 rpm.
7) Co-extrusion: The API loaded polymer pellets were further extruded as the core material with ethylene-vinyl acetate co-polymer (VA content 18% w/w) pellets as skin material through a coaxial extrusion set up. The coaxial extrusion set up comprises two extruders, two melt pumps, a co-extrusion die and connecting tubes. The core extruder process temperatures were set at 130-150° C. The core material output was controlled by a melt pump set at 42%. The skin extruder process temperatures were set at 120-150° C. The skin material output was controlled by a melt pump set at 30 rpm. The core-sheath fibre came out of the co-extrusion die and was cooled down in a vertical water bath. The water temperature was set to 9° C.
8) Strand cutting: The fibre was pulled at 3.8 m/min by a dragging conveyor before being cut to 157 mm segments/strands. The fibre diameter with 4 mm was continuously controlled by a laser gauge.
9) Welding: the two strand ends were thermally bonded. On the welding equipment, the edges of the 157 mm strands are merged forcing the fiber to assume a torus conformation yielding a core-sheath intravaginal ring with an outside diameter of 54 mm, an inside diameter of 46 mm and a cross sectional diameter of 4 mm.

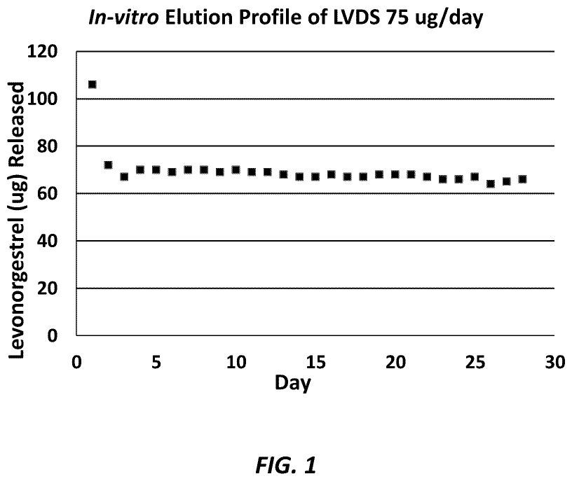

FIGS. 1, 2 and 3 and tables 2 to 4 below show the daily In-vitro Elution profile for the above Levonorgestrel intravaginal rings of 75, 125 and 150 g/day. The in-vitro release rate of levonorgestrel for example 1 was determined by immersing the samples in 200 mL (for day 1) and 100 mL (for day 2-28) of aqueous solution of 0.2M sodium acetate buffer with 1.0% sodium lauryl sulfate (SLS) surfactant and adjusted to pH 4.2 at 37 C under continuous stirring at 60 rpm. The levonorgestrel concentration was determined daily by HPLC using a Waters XBridge C18 column, flow rate of 1.0 ml/min, and an injection volume of 25 μL. Detection was carried out by UV detection at 240 nm.

TABLE 2
Individual Data, In-vitro Elution Profile, LVDS 75 μg/day
LNG 0.075 mg/day Vaginal Delivery System

| | Time | | | | | | | | | |
|---|---|---|---|---|---|---|---|---|---|---|
| | LNG (μg) Released | | | | | | | | | |
| Day | V1 | V2 | V3 | V4 | V5 | V6 | Mean | Min | Max | SD |
| 1 | 103 | 107 | 114 | 110 | 101 | 102 | 106 | 101 | 114 | 5.2 |
| 2 | 71 | 72 | 76 | 74 | 70 | 70 | 72 | 70 | 76 | 2.6 |
| 3 | 64 | 67 | 71 | 68 | 67 | 68 | 67 | 64 | 71 | 2.2 |
| 4 | 68 | 69 | 74 | 72 | 67 | 68 | 70 | 67 | 74 | 2.7 |
| 5 | 69 | 70 | 72 | 71 | 67 | 69 | 70 | 67 | 72 | 1.8 |
| 6 | 69 | 68 | 74 | 70 | 67 | 66 | 69 | 66 | 74 | 2.7 |
| 7 | 69 | 69 | 73 | 71 | 67 | 69 | 70 | 67 | 73 | 1.9 |
| 8 | 69 | 68 | 74 | 72 | 66 | 68 | 70 | 66 | 74 | 2.9 |
| 9 | 68 | 69 | 72 | 72 | 66 | 66 | 69 | 66 | 72 | 2.7 |
| 10 | 69 | 68 | 73 | 71 | 69 | 68 | 70 | 68 | 73 | 2.0 |
| 11 | 68 | 68 | 74 | 70 | 67 | 66 | 69 | 66 | 74 | 2.9 |
| 12 | 67 | 68 | 73 | 70 | 69 | 67 | 69 | 67 | 73 | 2.3 |
| 13 | 67 | 66 | 73 | 69 | 67 | 67 | 68 | 66 | 73 | 2.7 |
| 14 | 66 | 66 | 71 | 69 | 65 | 66 | 67 | 65 | 71 | 2.5 |
| 15 | 65 | 67 | 72 | 70 | 66 | 64 | 67 | 64 | 72 | 2.9 |
| 16 | 66 | 68 | 72 | 70 | 66 | 65 | 68 | 65 | 72 | 2.7 |
| 17 | 67 | 67 | 72 | 68 | 65 | 64 | 67 | 64 | 72 | 2.6 |
| 18 | 66 | 67 | 71 | 70 | 66 | 65 | 67 | 65 | 71 | 2.3 |
| 19 | 65 | 67 | 71 | 69 | 66 | 67 | 68 | 65 | 71 | 2.1 |
| 20 | 66 | 67 | 71 | 68 | 67 | 65 | 68 | 65 | 71 | 2.0 |
| 21 | 69 | 67 | 71 | 69 | 69 | 65 | 68 | 65 | 71 | 2.2 |
| 22 | 65 | 67 | 71 | 70 | 65 | 63 | 67 | 63 | 71 | 3.2 |
| 23 | 65 | 65 | 70 | 67 | 65 | 65 | 66 | 65 | 70 | 2.1 |
| 24 | 64 | 66 | 72 | 69 | 64 | 64 | 66 | 64 | 72 | 3.3 |
| 25 | 67 | 65 | 70 | 69 | 67 | 66 | 67 | 65 | 70 | 2.1 |
| 26 | 65 | 63 | 69 | 66 | 61 | 61 | 64 | 61 | 69 | 3.1 |
| 27 | 63 | 63 | 69 | 67 | 63 | 64 | 65 | 63 | 69 | 2.6 |
| 28 | 65 | 65 | 69 | 67 | 65 | 64 | 66 | 64 | 69 | 2.0 |
| Day 1 | 103 | 107 | 114 | 110 | 101 | 102 | 106 | 101 | 114 | 5.2 |
| Day 2-27 | 67 | 67 | 72 | 70 | 66 | 66 | 68 | 66 | 72 | 2.4 |
| Day 28 | 65 | 65 | 69 | 67 | 65 | 64 | 66 | 64 | 69 | 2.0 |

TABLE 3
Individual Data, In-vitro Elution Profile, LVDS 125 μg/day
LNG 0.125 mg/day Vaginal Delivery System

| | Time | | | | | | | | | |
|---|---|---|---|---|---|---|---|---|---|---|
| | LNG (μg) Released | | | | | | | | | |
| Day | V1 | V2 | V3 | V4 | V5 | V6 | Mean | Min | Max | SD |
| 1 | 148 | 146 | 148 | 156 | 151 | 146 | 149 | 146 | 156 | 3.8 |
| 2 | 128 | 127 | 127 | 130 | 126 | 124 | 127 | 124 | 130 | 2.1 |
| 3 | 127 | 126 | 124 | 128 | 127 | 122 | 126 | 122 | 128 | 2.4 |
| 4 | 126 | 124 | 123 | 130 | 127 | 124 | 126 | 123 | 130 | 2.4 |
| 5 | 122 | 125 | 124 | 126 | 126 | 123 | 124 | 122 | 126 | 1.5 |
| 6 | 122 | 124 | 124 | 129 | 123 | 124 | 124 | 122 | 129 | 2.4 |
| 7 | 123 | 125 | 123 | 128 | 126 | 122 | 125 | 122 | 128 | 2.3 |
| 8 | 123 | 124 | 122 | 129 | 127 | 122 | 125 | 122 | 129 | 2.7 |
| 9 | 121 | 123 | 121 | 127 | 124 | 121 | 123 | 121 | 127 | 2.3 |
| 10 | 123 | 122 | 122 | 127 | 124 | 121 | 123 | 121 | 127 | 2.1 |
| 11 | 122 | 123 | 122 | 128 | 124 | 122 | 124 | 122 | 128 | 2.5 |
| 12 | 121 | 124 | 121 | 125 | 123 | 121 | 123 | 121 | 125 | 1.5 |
| 13 | 122 | 124 | 121 | 128 | 123 | 121 | 123 | 121 | 128 | 2.4 |
| 14 | 120 | 121 | 118 | 123 | 120 | 116 | 120 | 116 | 123 | 2.4 |
| 15 | 120 | 118 | 119 | 122 | 121 | 118 | 120 | 118 | 122 | 1.7 |
| 16 | 118 | 121 | 117 | 125 | 120 | 118 | 120 | 117 | 125 | 2.7 |
| 17 | 116 | 118 | 116 | 123 | 120 | 117 | 118 | 116 | 123 | 2.9 |
| 18 | 117 | 120 | 119 | 122 | 120 | 117 | 119 | 117 | 122 | 2.2 |
| 19 | 117 | 117 | 119 | 122 | 118 | 117 | 118 | 117 | 122 | 1.9 |
| 20 | 116 | 115 | 115 | 119 | 115 | 117 | 116 | 115 | 119 | 1.7 |
| 21 | 111 | 112 | 113 | 114 | 114 | 113 | 113 | 111 | 114 | 1.0 |
| 22 | 110 | 111 | 110 | 118 | 113 | 109 | 112 | 109 | 118 | 3.2 |
| 23 | 109 | 112 | 110 | 113 | 111 | 108 | 110 | 108 | 113 | 1.9 |
| 24 | 106 | 109 | 106 | 110 | 108 | 108 | 108 | 106 | 110 | 1.5 |
| 25 | 106 | 107 | 105 | 111 | 106 | 106 | 107 | 105 | 111 | 2.1 |
| 26 | 105 | 106 | 104 | 108 | 106 | 104 | 106 | 104 | 108 | 1.8 |
| 27 | 102 | 103 | 107 | 109 | 104 | 104 | 105 | 102 | 109 | 2.5 |
| 28 | 99 | 101 | 102 | 106 | 101 | 101 | 102 | 99 | 106 | 2.4 |
| Day 1 | 148 | 146 | 148 | 156 | 151 | 146 | 149 | 146 | 156 | 3.8 |
| Day 2-27 | 117 | 118 | 117 | 122 | 119 | 117 | 119 | 117 | 122 | 1.9 |
| Day 28 | 99 | 101 | 102 | 106 | 101 | 101 | 102 | 99 | 106 | 2.4 |

TABLE 4
Individual Data, In-vitro Elution Profile, LVDS 150 μg/day
LNG 0.150 mg/day Vaginal Delivery System

| | Time | | | | | | | | | |
|---|---|---|---|---|---|---|---|---|---|---|
| | LNG (μg) Released | | | | | | | | | |
| Day | V1 | V2 | V3 | V4 | V5 | V6 | Mean | Min | Max | SD |
| 1 | 202 | 193 | 192 | 192 | 206 | 187 | 195 | 187 | 206 | 7.2 |
| 2 | 183 | 178 | 176 | 190 | 187 | 172 | 181 | 172 | 190 | 6.8 |
| 3 | 181 | 175 | 170 | 173 | 179 | 165 | 174 | 165 | 181 | 6.0 |
| 4 | 182 | 176 | 173 | 188 | 185 | 168 | 179 | 168 | 188 | 7.6 |
| 5 | 185 | 174 | 177 | 185 | 183 | 164 | 178 | 164 | 185 | 8.3 |
| 6 | 180 | 172 | 173 | 172 | 182 | 162 | 174 | 162 | 182 | 7.0 |
| 7 | 178 | 174 | 174 | 184 | 178 | 165 | 176 | 165 | 184 | 6.1 |
| 8 | 178 | 167 | 166 | 168 | 174 | 163 | 169 | 163 | 178 | 5.6 |
| 9 | 170 | 165 | 162 | 168 | 172 | 160 | 166 | 160 | 172 | 4.9 |
| 10 | 166 | 159 | 162 | 177 | 173 | 157 | 166 | 157 | 177 | 8.0 |
| 11 | 169 | 157 | 164 | 171 | 167 | 155 | 164 | 155 | 171 | 6.3 |
| 12 | 166 | 159 | 158 | 169 | 166 | 153 | 162 | 153 | 169 | 6.2 |
| 13 | 164 | 155 | 156 | 154 | 165 | 153 | 158 | 153 | 165 | 5.0 |
| 14 | 150 | 144 | 145 | 157 | 155 | 140 | 148 | 140 | 157 | 6.5 |
| 15 | 155 | 148 | 152 | 160 | 154 | 144 | 152 | 144 | 160 | 5.6 |
| 16 | 152 | 148 | 146 | 150 | 152 | 144 | 149 | 144 | 152 | 3.3 |
| 17 | 150 | 144 | 146 | 154 | 150 | 139 | 147 | 139 | 154 | 5.4 |
| 18 | 147 | 139 | 140 | 154 | 148 | 138 | 144 | 138 | 154 | 6.3 |
| 19 | 144 | 138 | 139 | 149 | 147 | 138 | 143 | 138 | 149 | 4.9 |
| 20 | 145 | 141 | 140 | 142 | 146 | 135 | 142 | 135 | 146 | 4.1 |
| 21 | 140 | 137 | 138 | 140 | 144 | 131 | 138 | 131 | 144 | 4.1 |
| 22 | 139 | 131 | 134 | 144 | 140 | 129 | 136 | 129 | 144 | 5.6 |
| 23 | 135 | 130 | 130 | 137 | 137 | 127 | 133 | 127 | 137 | 4.0 |
| 24 | 134 | 128 | 131 | 134 | 137 | 126 | 132 | 126 | 137 | 4.1 |
| 25 | 132 | 124 | 129 | 136 | 134 | 126 | 130 | 124 | 136 | 4.6 |
| 26 | 126 | 122 | 121 | 128 | 129 | 118 | 124 | 118 | 129 | 4.5 |
| 27 | 125 | 120 | 124 | 127 | 127 | 121 | 124 | 120 | 127 | 3.0 |
| 28 | 123 | 118 | 119 | 126 | 128 | 119 | 122 | 118 | 128 | 4.1 |
| Day 1 | 202 | 193 | 192 | 192 | 206 | 187 | 195 | 187 | 206 | 7.2 |
| Day 2-27 | 157 | 150 | 151 | 158 | 158 | 146 | 153 | 146 | 158 | 5.0 |
| Day 28 | 123 | 118 | 119 | 126 | 128 | 119 | 122 | 118 | 128 | 4.1 |

### Example 2

A Multi-center, phase 2, open-label, randomized clinical trial to evaluate the inhibition of ovulation of 3 dosing strengths of levonorgestrel (LNG) vaginal delivery system (VDS), releasing during 28 days in continuous regimen, versus desogestrel (Cerazet®) was performed as further detailed here below:

### 1. Study Design

### 1.1. Objectives

### Primary Objective:

Assess the inhibition of ovulation measured by the ovarian activity (follicular growth, estradiol, and progesterone serum concentrations) of levonorgestrel (LNG) vaginal delivery system (VDS) in treatment cycles (TCs) 1 and 2 as compared to desogestrel (Cerazet).

### Secondary Objectives:

Assess the influence of LNG VDS on the cervical mucus and the endometrial thickness.
Assess the impact of LNG VDS on the blood levels of sexual hormones.
Assess the safety and tolerability of LNG VDS, and the return of ovulation in a post-treatment cycle.

### 1.2. Methodology

Multi-center, open-label, randomized, phase 2 clinical trial to evaluate the inhibition of ovulation of 3 different dosing strengths (75 μg/day, 125 μg/day and 150 μg/day) of LNG VDS released during 28 days in continuous regimen versus desogestrel (Cerazet) in healthy female subjects aged 18-35 years.

The study consists of 4 phases:
Screening phase (minimum of 4 weeks and maximum of 8 weeks if washout cycle was needed).
Pre-treatment cycle (28 days) and randomization.
Treatment cycle consisting of 56 treatment days (2 cycles, 28 days per cycle).
Post-treatment cycle (28 days).

Overall, 268 subjects were screened, of whom 137 were randomized and 130 started the study treatment (safety analysis set or SAS). The efficacy was assessed in a total of 128 participants comprising the full analysis set or FAS, and in 118 participants comprising the per protocol set or PP. For the PK/PD analysis, 55 participants comprised the PK population (14 out of them had a BMI ≥30 kg/m2 and 41 had a BMI between ≥18 and <30 kg/m2).

### 1.3. Test Product, Dose and Mode of Administration

LNG VDS for vaginal administration was used. Each LNG VDS contained about 10 mg of LNG that was designed to release 75 μg/day, 125 μg/day, or 150 μg/day of LNG. One LNG VDS was to last for 28 continuous days.

### 1.4. Duration of Treatment

The treatment was performed for 56 days, i.e., two consecutive Treatment Cycles (TC1 and TC2) each of 28 days.

### 1.5. Reference Therapy, Dose and Mode of Administration

As a reference Desogestrel (Cerazet) 75 μg film-coated tablets were used and administrated orally for 28 continuous days in each cycle.

### 2. Criteria for Evaluation

### 2.1. Primary Efficacy

The inhibition of ovulation was determined by the calculation of the Hoogland Score, which combined the measurement of follicle size in mm by TVU and progesterone/estradiol serum concentration in nmol/L (in case an ovulation was suspected sonographically in TC1 or TC2, it was confirmed by blood progesterone levels and reflected by Landgren Score).

Primary endpoint analysis: the inhibition of ovulation (Yes, No), was analyzed through a logistic regression model to compare inhibition of ovulation across treatment groups in which BMI was included as covariate. Adjusted odds ratios (ORs) with their two-sided 95% CI were calculated. No hypothesis testing was performed.

### 2.2. Secondary Efficacy

Insler Score in the pre-cycle, TC1 and TC2 and posttreatment cycle was assessed whenever follicles had a diameter of >13 mm
Endometrial thickness was followed throughout all cycles by TVUs
Serum levels of follicle stimulating hormone (FSH) and luteinizing hormone (LH), estradiol, progesterone and SHBG were analyzed.
The return of ovulation was evaluated in a posttreatment cycle

Secondary endpoint analysis: secondary efficacy parameters were described with appropriate descriptive statistics for dichotomous, categorical, or continuous variables by treatment group and cycle. No hypothesis testing was performed.

### 2.3. Safety

Vital signs
Laboratory analysis
Incidence of adverse events (AEs)
Vaginal bleeding pattern were evaluated by means of daily diary entries by the subjects, therefore assessing the cycle control and occurrence of intermediate bleedings.

### 2.4. PK Analysis

PK parameters of LNG were correlated to SHBG levels and the inhibition of ovulation within a subgroup of subjects receiving LNG from the total randomized composed of 12 subjects with BMI ≥18 and <30 kg/m2 and 7 subjects with BMI ≥30 kg/m2 per treatment arm (75 μg/day, 125 μg/day or 150 μg/day).

Pharmacokinetic parameters of LNG were determined to correlate them with the inhibition of ovulation and SHBG levels in a subgroup of 55 subjects that were using the LNG VDS, comprising 41 subjects with a BMI ≥18 and <30 kg/m2 and 14 subjects with BMI ≥30 kg/m2.

### 3. Statistical Method

The analysis of the present study was exploratory and primarily made use of descriptive statistical methods. In addition, exploratory statistical testing and modelling was used to highlight interesting aspects of the data. Descriptive statistics presented for continuous variables (Landgren Score, Insler Score, endometrial thickness, pituitary hormones, bleeding pattern, demography and baseline characteristics as well as safety parameters) were the number of subjects (n), mean (mean), median (median), standard deviation (SD), minimum (min) and maximum (max) and 1-3 quantiles at each scheduled visit.

Changes from baseline were presented with the 95% confidence interval (CI) when applicable and were calculated as absolute changes as assessment visit value minus baseline value. For categorical variables including binary variables, the absolute (n) and relative frequency (%) along with number of missing data were summarized for each category at each scheduled visit.

### 4. Results and Analysis of Efficacy

### 4.1. Primary Efficacy Assessment

### 4.1.1. Inhibition of Ovulation

The results obtained regarding the inhibition of ovulation by treatment cycle and by treatment group is presented in Table 5 for the FAS. In TC1, 127 (99.2%) participants showed inhibition of ovulation in all treatment groups and only 1 participant with BMI ≥18 and <30 kg/m2 who was treated with Cerazet did not show inhibition of ovulation. In TC2, inhibition of ovulation was confirmed for all participants regardless of the treatment group and BMI.

TABLE 5
Inhibition of Ovulation by Treatment Cycle - Logistic Regression Model

| | All (N = 128) | | | | All (N = 23) | | | |
|---|---|---|---|---|---|---|---|---|
| | | | Subjects with | | Total | | Subjects with | |
| | Total | | ovulation | | analyzed | | ovulation | |
| | analyzed | | inhibition | | n (%) by | | inhibition n (%) by | |
| | n (%) | | n(%) [1] [2] | | BMI ≥30 | | BMI ≥30 [1] [2] | |
| Treatment Cycle 1 | 128 | | 127 | (99.2%) | 23 | | 23 | (100.0%) |
| Cerazet | 33 | (25.8%) | 32 | (97.0%) | 6 | (26.1%) | 6 | (100.0%) |
| LNG VDS 75 | 30 | (23.4%) | 30 | (100.0%) | 4 | (17.4%) | 4 | (100.0%) |
| LNG VDS 125 | 34 | (26.6%) | 34 | (100.0%) | 7 | (30.4%) | 7 | (100.0%) |
| LNG VDS 150 | 31 | (24.2%) | 31 | (100.0%) | 6 | (26.1%) | 6 | (100.0%) |
| BMI | | | | | | | | |
| 18 ≤ BMI < 30 | 105 | (82.0%) | 104 | (99.0%) | | | | |
| BMI ≥30 | 23 | (18.0%) | 23 | (100.0%) | | | | |
| Treatment Cycle 2 | 125 | | 125 | (100.0%) | 22 | | 22 | (100.0%) |
| Cerazet | 31 | (24.8%) | 31 | (100.0%) | 6 | (27.3%) | 6 | (100.0%) |
| LNG VDS 75 | 30 | (24.0%) | 30 | (100.0%) | 4 | (18.2%) | 4 | (100.0%) |
| LNG VDS 125 | 33 | (26.4%) | 33 | (100.0%) | 6 | (27.3%) | 6 | (100.0%) |
| LNG VDS 150 | 31 | (24.8%) | 31 | (100.0%) | 6 | (27.3%) | 6 | (100.0%) |
| BMI | | | | | | | | |
| 18 ≤ BMI < 30 | 103 | (82.4%) | 103 | (100.0%) | | | | |
| BMI ≥30 | 22 | (17.6%) | 22 | (100.0%) | | | | |
| Pooled [3] | 128 | | 127 | (99.2%) | 23 | | 23 | (100.0%) |
| Cerazet | 33 | (25.8%) | 32 | (97.0%) | 6 | (26.1%) | 6 | (100.0%) |
| LNG VDS 75 | 30 | (23.4%) | 30 | (100.0%) | 4 | (17.4%) | 4 | (100.0%) |
| LNG VDS 125 | 34 | (26.6%) | 34 | (100.0%) | 7 | (30.4%) | 7 | (100.0%) |
| LNG VDS 150 | 31 | (24.2%) | 31 | (100.0%) | 6 | (26.1%) | 8 | (100.0%) |
| BMI | | | | | | | | |
| 18 ≤ BMI < 30 | 105 | (82.0%) | 104 | (99.0%) | | | | |
| BMI ≥30 | 23 | (18.0%) | 23 | (100.0%) | | | | |

Adjusted Odds Ratio (95% CI) not applicable
Note:
Parameters for logistic regression model using BMI as covariare. Adjusted Odds Ratio for Ovulation: Yes vs No.
Note:
For each treatment cycle, the highest score result over the treatment was considered for the analysis.
[1]: Inhibition of ovulation assessed as Hoogland score of 1-4 (both included)
[2]: % calculated using total number of Subjects of each factor category.
[3]: Pooled comprehends results of Treatment Cycle 1 and Treatment Cycle 2. Pooled Hoogland score considered as ovulation whenever there was ovulation in any cycle independently of the other cycle result (no ovulation/missing). Pooled Hoogland score considered as missing whenever there was missing data in both cycles. In case there was no ovulation in a cycle and missing data in the other, it was considered globally as no ovulation.

### 4.1.2. Hoogland and Landgren Score

The number and percentage of participants who had none, residual or high ovarian activity by treatment group and by BMI group in each treatment cycle in the FAS is presented in Table 6.

TABLE 6
Hoogland Score by Treatment Cycle - Sensitivity Analysis by Body Mass Index (Proportional Odds Model)

| | All (N = 128) | | | | All BMI ≥ 30 (N = 23) | | | |
|---|---|---|---|---|---|---|---|---|
| | Total | None | Residual | High | Total | None | Residual | High |
| | analyzed | n (%) | n (%) | n (%) | analyzed | n (%) | n (%) | n (%) |
| | n (%) | [1] [2] | [3] [3] | [1] [4] | n (%) | [1] [2] | [1] [3] | [1] [4] |
| Treatment | 128 | 88 (68.8%) | 39 (30.5%) | 1 (0.8%) | 23 | 11 (47.8%) | 12 (52.2%) | 0 (0.0%) |
| Cycle 1 | | | | | | | | |
| Treatment | | | | | | | | |
| Cerazet | 33 (25.8%) | 19 (57.6%) | 13 (39.4%) | 1 (3.0%) | 6 (26.1%) | 1 (16.7%) | 5 (83.3%) | 0 (0.0%) |
| LNG VDS 75 | 30 (23.4%) | 17 (56.7%) | 13 (43.3%) | 0 (0.0%) | 4 (17.4%) | 2 (50.0%) | 2 (50.0%) | 0 (0.0%) |
| LNG VDS 125 | 34 (26.6%) | 26 (76.5%) | 8 (23.5%) | 0 (0.0%) | 7 (30.4%) | 3 (42.9%) | 4 (57.1%) | 0 (0.0%) |
| LNG VDS 150 | 31 (24.2%) | 26 (83.9%) | 5 (16.1%) | 0 (0.0%) | 6 (26.1%) | 5 (83.3%) | 1 (16.7%) | 0 (0.0%) |
| BMI | | | | | | | | |
| 18 ≤ BMI < 30 | 105 (82.0%) | 77 (73.3%) | 27 (25.7%) | 1 (1.0%) | | | | |
| BMI ≥ 30 | 23 (18.0%) | 11 (47.8%) | 12 (52.2%) | 0 (0.0%) | | | | |
| Treatment | 125 | 89 (71.2%) | 36 (28.3%) | 0 (0.0%) | 22 | 15 (68.2%) | 7 (31.8%) | 0 (0.0%) |
| Cycle 2 | | | | | | | | |
| Treatment | | | | | | | | |
| Cerazet | 31 (24.8%) | 15 (48.4%) | 16 (51.6%) | 0 (0.0%) | 6 (27.3%) | 2 (33.3%) | 4 (66.7%) | 0 (0.0%) |
| LNG VDS 75 | 30 (24.0%) | 19 (63.3%) | 11 (36.7%) | 0 (0.0%) | 4 (18.2%) | 3 (75.0%) | 1 (25.0%) | 0 (0.0%) |
| LNG VDS 125 | 33 (26.4%) | 28 (84.8%) | 5 (15.2%) | 0 (0.0%) | 6 (27.3%) | 5 (83.3%) | 1 (16.7%) | 0 (0.0%) |
| LNG VDS 150 | 31 (24.8%) | 27 (87.1%) | 4 (12.9%) | 0 (0.0%) | 6 (27.3%) | 5 (83.3%) | 1 (16.7%) | 0 (0.0%) |
| BMI | | | | | | | | |
| 18 ≤ BMI < 30 | 103 (82.4%) | 74 (71.8%) | 29 (28.2%) | 0 (0.0%) | | | | |
| BMI ≤ 30 | 22 (17.6%) | 15 (68.2%) | 7 (31.8%) | 0 (0.0%) | | | | |
| Pooled [5] | 128 | 79 (61.7%) | 48 (37.5%) | 1 (0.8%) | 23 | 11 (47.8%) | 12 (52.2%) | 0 (0.0%) |
| Treatment | | | | | | | | |
| Cerazet | 33 (25.8%) | 14 (42.4%) | 18 (54.5%) | 1 (3.0%) | 6 (26.1%) | 1 (16.7%) | 5 (83.3%) | 0 (0.0%) |
| LNG VDS 75 | 30 (23.4%) | 15 (50.0%) | 15 (50.0%) | 0 (0.0%) | 4 (17.4%) | 2 (50.0%) | 2 (50.0%) | 0 (0.0%) |
| LNG VDS 125 | 34 (26.6%) | 24 (70.6%) | 10 (29.4%) | 0 (0.0%) | 7 (30.4%) | 3 (42.9%) | 4 (57.1%) | 0 (0.0%) |
| LNG VDS 150 | 31 (24.2%) | 26 (83.9%) | 5 (16.1%) | 0 (0.0%) | 6 (26.1%) | 5 (83.3%) | 1 (16.7%) | 0 (0.0%) |
| BMI | | | | | | | | |
| 18 ≤ BMI < 30 | 105 (82.0%) | 68 (64.8%) | 36 (34.3%) | 1 (1.0%) | | | | |
| BMI ≥ 30 | 23 (18.0%) | 11 (47.8%) | 12 (52.2%) | 0 (0.0%) | | | | |

Adjusted Odds Ratio (95% CI) not applicable
Note:
Parameters for multinomial logistic regression proportional odds model using BMI as covariate. High ovarian activity including ovulation was the reference group for Hoogland Score.
Note:
For each treatment cycle, the highest score result over the treatment was considered for the analysis.
[1] % calculated using total number of Subjects of each factor category.
[2] No or minimum ovarian activity: Hoogland score 1 or 2.
[3] Residual ovarian activity: Hoogland score 3 or 4
[4] High ovarian activity including adding ovulation: Hoogland score 5 or 6
[5] Pooled comprehends results of Treatment Cycle 1 and Treatment Cycle 2. Pooled Hoogland score considered as ovulation whenever there was ovulation in any cycle independently of the other cycle result (no ovulation/missing). Pooled Hoogland score considered as missing whenever there was missing data in both cycles. In case there was no ovulation in a cycle and missing data in the data, it was considered globally as no ovulation.

In TC1, a total of 88 (68.8%) participants had no or minimum ovarian activity (Hoogland score of 1 or 2) corresponding to 19 (57.6%), 17 (56.7%), 26 (76.5%) and 26 (83.9%) participants treated with Cerazet, LNG VDS 75, 125 and 150, respectively; and 39 (30.5%) had residual ovarian activity (Hoogland score of 3 or 4) corresponding to 3 (39.4%), 13 (43.3%), 8 (23.5%), 5 (16.1%) participants treated with Cerazet, LNG VDS 75, 125 and 150, respectively.

There was only 1 (0.8%) participant, who was treated with Cerazet, who had a high ovarian activity (Hoogland score from 5 to 6) during TC1.

In the ANCOVA model performed, the LS mean (95% CI) of the differences considering Cerazet as the reference group was −0.2 (0.30) (−0.8; 0.4) for LNG VDS 75, −0.6 (0.29) (−1.1; 0.0) for LNG VDS 125 and −0.7 (0.29) (−1.3; −0.2) for LNG VDS 150.

The assessment of the Hoogland score by BMI group showed that no or minimum ovarian activity was observed in 77 (73.3%) and 11 (47.8%) participants with a BMI ≥18 and <30 kg/m2 and participants with a BMI ≥30 kg/m2, respectively, and residual ovarian activity was observed in 27 (25.7%) and 11 (47.8%) participants with a BMI ≥18 and <30 kg/m2 and participants with a BMI ≥30 kg/m2, respectively.

In the ANCOVA model performed, the LS mean (95% CI) of the differences considering the group of participants with BMI ≥18 and <30 kg/m2 was 0.8 (0.27) (0.2; 1.3) for participants with BMI ≥30 kg/m2.

In the group of participants with a BMI ≥30 kg/m2, 1 (16.7%), 2 (50.0%), 3 (42.9%) and 5 (83.3%) participants treated with Cerazet, LNG VDS 75, 125 and 150 had no or minimum ovarian activity; and 5 (83.3%), 2 (50.0%), 4 (57.1%) and 1 (16.7%) participant(s) treated with Cerazet, LNG VDS 75, 125 and 150, respectively, had residual ovarian activity. There were no participants with BMI ≥30 kg/m2 who reported high ovarian activity during TC1.

In TC2, a total of 89 (71.2%) and 36 (28.8%) participants had no or minimum ovarian activity (Hoogland score of 1 or 2) and residual ovarian activity (Hoogland score of 3 or 4), respectively.

By treatment group, 15 (48.4%), 19 (63.3%), 28 (84.8%) and 27 (87.1%), participants treated with Cerazet, LNG VDS 75, 125 and 150, respectively, had no or minimum ovarian activity; and 16 (51.6%), 11 (36.7%), 5 (15.2%) and 4 (12.9%) participants treated with Cerazet, LNG VDS 75, 125 and 150, respectively, had residual ovarian activity.

There were no participants who had a high ovarian activity (Hoogland score from 5 to 6) during TC1. In the ANCOVA model performed, the LS mean (95% CI) of the differences considering Cerazet as the reference group was −0.4 (0.29) (−0.9; 0.2) for LNG VDS 75, −0.8 (0.29) (−1.4; −0.3) for LNG VDS 125 and −1.0 (0.29) (−1.6; −0.5) for LNG VDS 150.

The assessment of the Hoogland score by BMI group showed that no or minimum ovarian activity was observed in 74 (71.8%) and 15 (68.2%) participants with a BMI ≥18 and <30 kg/m2 and participants with a BMI ≥30 kg/m2, respectively, and residual ovarian activity was observed in 29 (28.2%) and 7 (31.8%) participants with a BMI ≥18 and <30 kg/m2 and participants with a BMI ≥30 kg/m2, respectively.

In the ANCOVA model performed, the LS mean (95% CI) of the differences considering the group of participants with BMI ≥18 and <30 kg/m2 was 0.2 (0.27) (−0.4-0.7) for participants with BMI ≥30 kg/m2.

In the group of participants with a BMI ≥30 kg/m2, 2 (33.3%), 3 (75.0%), 5 (83.3%) and 5 (83.3%) participants treated with Cerazet, LNG VDS 75, 125 and 150 had no or minimum ovarian activity; and 4 (66.7%), 1 (25.0%), 1 (16.7%) and 1 (16.7%) participants treated with Cerazet, LNG VDS 75, 125 and 150, respectively, had residual ovarian activity. There were no participants with BMI ≥30 kg/m2 who reported high ovarian activity during TC1.

Landgren assessment was performed in 43 (33.6%) participants (when ovulation was suspected by the follicle size measured by TVU) of the FAS during TC1 and TC2 (10 [33.3%] participants in the LNG VDS 75 treatment group, 10 [29.4%] in the LNG VDS 125, 10 [32.3%] in the LNG VDS 150 and 13 [39.4%] in the Cerazet treatment group). However, this test was positive only in 1 (0.8%) participant in the Cerazet treatment group. There were no participants treated with LNG VDS who reported a positive Landgren test.

### 4.1.3. Follicle Size

Follicle size by treatment cycle has been assessed in each treatment group (Table 4). During TC1, the mean (SD) size of the largest follicle was 10.2 (3.8) mm assessed in 117 participants from the FAS. Numerical differences were observed between treatment groups: 11.7 (4.6) mm, 11.2 (4.2) mm, 9.4 (3.4) mm and 8.6 (2.1) mm in the Cerazet, LNG VDS 75, 125 and 150 treatment groups, respectively.

In TC2, the mean (SD) size of the largest follicle was 9.8 (3.2) mm assessed in 111 participants from the FAS. Numerical differences were also observed between treatment groups: 12.0 (4.1) mm, 10.4 (3.5) mm, 8.7 (1.8) mm and 8.2 (1.3) mm in the Cerazet, LNG VDS 75, 125 and 150 treatment groups, respectively.

Among participants with a BMI ≥30 kg/m2 (Table 12), the mean (SD) largest follicle size during TC1 was 11.5 (4.2) mm (14.5 [5.5] mm, 11.0 [4.3] mm, 11.3 [3.2] mm and 9.2 [2.8] mm in the Cerazet, LNG VDS 75, 125 and 150 treatment groups, respectively) which was slightly higher compared to the whole population. In TC2, the mean (SD) largest follicle size decreased to 9.8 (3.3) mm (12.5 [4.8] mm, 9.4 [3.1] mm, 8.8 [0.6] mm and 8.3 [1.4] mm in the Cerazet, LNG VDS 75, 125 and 150 treatment groups, respectively), which was very similar to the value obtained in the whole population.

TABLE 7
Follicle Size by Treatment Cycle

| | LNG VDS 75 | LNG VDS 125 | LNG VDS 150 | Cerazet | All |
|---|---|---|---|---|---|
| | (N = 30/n = 4) | (N = 34/n = 7) | (N = 31/n = 6) | (N = 33/n = 6) | (N = 128/n = 23) |
| Treatment cycle 1 | | | | | |
| Size of largest | | | | | |
| follicle size (mm) | | | | | |
| Number | 26 | 32 | 29 | 30 | 117 |
| Mean (SD) | 11.2 (4.2) | 9.4 (3.4) | 8.6 (2.1) | 11.7 (4.6) | 10.2 (3.8) |
| Median | 9.8 | 8.6 | 7.8 | 10.5 | 9.0 |
| Q1:Q3 | 7.9:13.7 | 7.7:10.0 | 7.2:9.9 | 8.2:14.8 | 7.7:11.3 |
| Min:Max | 6.4:22.0 | 4.7:21.6 | 5.7:14.7 | 6.8:21.8 | 4.7:22.0 |
| Missing | 4 | 2 | 2 | 3 | 11 |
| Size of largest follicle | | | | | |
| size (mm) − BMI ≥ 30 | | | | | |
| Number | 4 | 7 | 6 | 6 | 23 |
| Mean (SD) | 11.0 (4.3) | 11.3 (3.2) | 9.2 (2.8) | 14.5 (5.5) | 11.5 (4.2) |
| Median | 10.6 | 10.0 | 8.1 | 16.0 | 9.7 |
| Q1:Q3 | 7.5:14.4 | 8.6:15.4 | 7.5:9.7 | 8.6:18.4 | 8.3:15.4 |
| Min:Max | 6.4:16.1 | 8.3:16.1 | 7.2:14.7 | 7.3:20.9 | 6.4:20.9 |
| Missing | 0 | 0 | 0 | 0 | 0 |
| Treatment cycle 2 | | | | | |
| Size of largest | | | | | |
| follicle size (mm) | | | | | |
| Number | 25 | 29 | 29 | 28 | 111 |
| Mean (SD) | 10.4 (3.5) | 8.7 (1.8) | 8.2 (1.3) | 12.0 (4.1) | 9.8 (3.2) |
| Median | 9.4 | 8.7 | 7.8 | 11.0 | 9.0 |
| Q1:Q3 | 8.4:12.3 | 7.2:9.6 | 7.0:9.1 | 8.7:15.1 | 7.6:10.4 |
| Min:Max | 6.0:22.8 | 6.0:13.9 | 6.2:11.8 | 6.6:22.2 | 6.0:22.8 |
| Missing | 5 | 5 | 2 | 5 | 17 |
| Size of largest follicle | | | | | |
| size (mm) − BMI ≥ 30 | | | | | |
| Number | 4 | 5 | 6 | 6 | 21 |
| Mean (SD) | 9.4 (3.1) | 8.8 (0.6) | 8.3 (1.4) | 12.5 (4.8) | 9.8 (3.3) |
| Median | 8.9 | 8.8 | 7.7 | 14.0 | 8.8 |
| Q1:Q3 | 7.3:11.6 | 8.7:8.8 | 7.6:9.5 | 6.7:15.1 | 7.6:10.5 |
| Min:Max | 6.4:13.6 | 8.1:9.7 | 7.0:10.5 | 6.8:18.6 | 6.4:18.6 |
| Missing | 0 | 2 | 0 | 0 | 2 |

N: total number of subjects.
n: number subjects with BMI ≥ 30

### 4.1.4. Serum Progesterone Levels

Serum progesterone levels were assessed from Day 3 to Day 27 every three days during TC1 and from Day 3 to Day 27 every three days and on Day 29 during TC2. The progesterone levels by visit and for each treatment group have been assessed in the whole FAS and among participants with a BMI≥30 kg/m2 as well as in the PP set.

In TC1, a higher serum progesterone levels were observed among participants treated with Cerazet compared to those treated with LNG VDS in any of its 3 releasing rates, mainly on Day 21 (2.3 [8.7] nmol/L, 0.9 [0.5] nmol/L, 0.7 [0.6] nmol/L and 0.6 [0.5] nmol/L in the Cerazet, LNG VDS 75, 125 and 150 treatment groups, respectively) and Day 24 (1.7 [6.1] nmol/L, 0.9 [0.5] nmol/L, 0.8 [0.7] nmol/L and 0.6 [0.5] nmol/L in the Cerazet, LNG VDS 75, 125 and 150 treatment groups, respectively).

In TC2, the mean progesterone levels were similar between treatment groups in all visits. In participants with BMI ≥30 kg/m2, no high variations were observed among treatment groups in both TC1 and TC2 by study visit, although serum progesterone levels were in general lower than those observed in the general population.

### 4.1.5. Serum Estradiol Levels

Serum estradiol levels were assessed from Day 3 to Day 27 every three days during TC1 and from Day 3 to Day 27 every three days and on Day 29 during TC2, see table 8.

TABLE 8
Estradiol Level by Treatment Cycle

| | LNG VDS 75 | | LNG VDS 125 | | LNG VDS 150 | | Cerazet | | All | |
|---|---|---|---|---|---|---|---|---|---|---|
| | (N = 30/n = 4) | | (N = 34 0 = 7) | | (N = 31/n = 6) | | (N = 33/n = 6) | | (N = 128/n = 23) | |
| Treatment cycle 1 | | | | | | | | | | |
| Mean Estradiol (pg/mL) | | | | | | | | | | |
| Number | 30 | | 34 | | 31 | | 33 | | 128 | |
| Mean (SD) | 59.3 | (40.2) | 37.3 | (21.2) | 32.1 | (12.1) | 97.1 | (84.3) | 56.6 | (54.6) |
| Median | 44.8 | | 32.8 | | 29.1 | | 56.4 | | 38.1 | |
| Q1:Q3 | 35.7:65.8 | | 24.7:40.9 | | 23.4:36.6 | | 38.1:128.9 | | 29.2:58.6 | |
| Min:Max | 20.7:216.4 | | 14.0:124.4 | | 16.7:74.8 | | 27.9:373.9 | | 14.0:373.9 | |
| Missing | 0 | | 0 | | 0 | | 0 | | 0 | |
| Mean Estradiol (pg/mL) | | | | | | | | | | |
| Number | 30 | | 34 | | 31 | | 33 | | 128 | |
| <20 | 0 | (0.0%) | 4 | (11.8%) | 3 | (9.7%) | 0 | (0.0%) | 7 | (5.5%) |
| [20-30[ | 2 | (6.7%) | 10 | (29.4%) | 14 | (45.2%) | 1 | (3.0%) | 27 | (21.1%) |
| [30-50] | 15 | (50.0%) | 16 | (47.1%) | 12 | (38.7%) | 13 | (39.4%) | 56 | (43.8%) |
| >50 | 13 | (43.3%) | 4 | (11.8%) | 2 | (6.5%) | 19 | (57.6%) | 38 | (29.7%) |
| Missing | 0 | | 0 | | 0 | | 0 | | 0 | |
| Mean Estradiol (pg/mL) - BMI ≥30 | | | | | | | | | | |
| Number | 4 | | 7 | | 6 | | 6 | | 23 | |
| Mean (SD) | 58.7 | (25.9) | 50.2 | (17.0) | 33.8 | (20.3) | 123.6 | (74.9) | 66.5 | (53.1) |
| Median | 51.5 | | 47.0 | | 27.2 | | 139.8 | | 42.9 | |
| Q1:Q3 | 40.0:77.4 | | 39.1:60.1 | | 23.8:28.2 | | 36.5:168.6 | | 34.0:83.8 | |
| Min:Max | 37.2:94.8 | | 34.0:83.8 | | 21.5:74.8 | | 34.4:222.2 | | 21.5:222.2 | |
| Missing | 0 | | 0 | | 0 | | 0 | | 0 | |
| Mean Estradiol (pg/mL) - BMI ≥30 | | | | | | | | | | |
| Number | 4 | | 7 | | 6 | | 6 | | 23 | |
| <20 | 0 | (0.0%) | 0 | (0.0%) | 0 | (0.0%) | 0 | (0.0%) | 0 | (0.0%) |
| [20-30[ | 0 | (0.0%) | 0 | (0.0%) | 5 | (83.3%) | 0 | (0.0%) | 5 | (21.7%) |
| [30-50] | 2 | (50.0%) | 5 | (71.4%) | 0 | (0.0%) | 2 | (33.3%) | 9 | (39.1%) |
| >50 | 2 | (50.0%) | 2 | (28.6%) | 1 | (16.7%) | 4 | (66.7%) | 9 | (39.1%) |
| Missing | 0 | | 0 | | 0 | | 0 | | 0 | |
| Treatment cycle 2 | | | | | | | | | | |
| Mean Estradiol (pg/mL) | | | | | | | | | | |
| Number | 30 | | 33 | | 31 | | 31 | | 125 | |
| Mean (SD) | 48.0 | (21.4) | 30.4 | (10.4) | 26.8 | (8.2) | 66.5 | (41.6) | 42.7 | (28.7) |
| Median | 43.6 | | 29.5 | | 25.7 | | 57.8 | | 34.7 | |
| Q1:Q2 | 29.2:58.8 | | 23.7:35.7 | | 19.3:33.6 | | 36.0:89.3 | | 25.4:45.7 | |
| Min:Max | 22.6:110.2 | | 5.5:57.7 | | 14.6:42.3 | | 22.5:218.8 | | 5.5:218.8 | |
| Mising | 0 | | 1 | | 0 | | 2 | | 3 | |
| Mean Estradiol (pg/mL) | | | | | | | | | | |
| Number | 30 | | 33 | | 31 | | 31 | | 125 | |
| <20 | 0 | (0.0%) | 4 | (12.1%) | 8 | (25.8%) | 0 | (0.0%) | 12 | (9.6%) |
| [20-30[ | 8 | (26.7%) | 13 | (39.4%) | 13 | (41.9%) | 2 | (6.5%) | 36 | (28.8% |
| [30-50] | 12 | (40.0%) | 14 | (42.4%) | 10 | (32.3%) | 13 | (41.9%) | 49 | (39.2%) |
| >50 | 10 | (33.3%) | 2 | (6.1%) | 0 | (0.0%) | 16 | (51.6%) | 28 | (22.4%) |
| Missing | 0 | | 1 | | 0 | | 2 | | 3 | |
| Mean Estradiol (pg/mL) - BMI ≥30 | | | | | | | | | | |
| Number | 4 | | 6 | | 6 | | 6 | | 22 | |
| Mean (SD) | 44.6 | (26.8) | 35.4 | (3.3) | 28.4 | (8.3) | 75.5 | (41.0) | 46.1 | (29.9 |
| Median | 36.8 | | 35.5 | | 25.9 | | 83.5 | | 34.8 | |
| Q1:Q3 | 25.5:63.7 | | 32.8:37.8 | | 23.2:33.6 | | 34.3:96.2 | | 26.4:45.3 | |
| Min:Max | 22.6:82.2 | | 30.9:40.0 | | 19.3:42.3 | | 22.5:132.6 | | 19.3:132.6 | |
| Missing | 0 | | 1 | | 0 | | 0 | | 1 | |
| Mean Estradiol (pg/mL) - BMI ≥30 | | | | | | | | | | |
| Number | 4 | | 6 | | 6 | | 6 | | 22 | |
| <20 | 0 | (0.0%) | 0 | (0.0%) | 1 | (16.7%) | 0 | (0.0%) | 1 | (4.5%) |
| [20-30[ | 2 | (50.0%) | 0 | (0.0%) | 3 | (50.0%) | 1 | (16.7%) | 6 | (27.3%) |
| [30-50] | 1 | (25.0%) | 6 | (100.0%) | 2 | (33.3%) | 1 | (16.7%) | 10 | (45.5%) |
| >50 | 1 | (25.0%) | 0 | (0.0%) | 0 | (0.0%) | 4 | (66.7%) | 5 | (22.7%) |

| | | | | | |
|---|---|---|---|---|---|
| Missing | 0 | 1 | 0 | 0 | 1 |

N: total number of subjects,
n: number of subjects with BMI ≥30

In TC1 the mean (SD) estradiol concentration in the FAS was 56.6 (54.6) pg/mL (97.1 [84.3] pg/mL, 59.3 [40.2] pg/mL, 37.3 [21.2] pg/mL and 32.1 [12.1] pg/mL in the Cerazet, LNG VDS 75, 125 and 150 treatment groups, respectively).

In the overall population 7 (5.5%) participants had a mean concentration of estradiol <20 μg/mL (0 [0.0%], 0 [0.0%], 4 [11.8%] and 3 [9.7%], in the Cerazet, LNG VDS 75, 125 and 150 treatment groups, respectively) and 27 (21.1%) participants had a mean estradiol concentration ≥20 and <30 μg/mL (1 [3.0%], 2 [6.7%], 10 [29.4%], 14 [45.2%] in the Cerazet, LNG VDS 75, 125 and 150 treatment groups, respectively).

In TC2 the mean (SD) estradiol concentration was 42.7 (28.7) pg/mL (66.5 [41.6] pg/mL, 48.0 [21.4] pg/mL, 30.4 [10.4] pg/mL and 26.8 [8.2] pg/mL in the Cerazet, LNG VDS 75, 125 and 150 treatment groups, respectively).

In the overall population, 12 (9.6%) participants had a mean concentration of estradiol <20 μg/mL (0 [0.0%], 0 [0.0%], 4 [12.1%] and 8 [25.8%] in the Cerazet, LNG VDS 75, 125 and 150 treatment groups, respectively) and 36 (28.8%) participants had a mean estradiol concentration ≥20 and <30 μg/mL (2 [6.5%], 8 [26.7%], 13 [39.4%] and 13 [41.9%] in the Cerazet, LNG VDS 75, 125 and 150 treatment groups, respectively).

In the group of participants with BMI ≥30 kg/m2 the mean (SD) estradiol concentration in TC1 was 66.5 (53.1) pg/mL (123.6 [74.9] pg/mL, 58.7 [25.9] pg/mL, 50.2 [17.0] pg/mL and 33.8 [20.3] pg/mL in the Cerazet, LNG VDS 75, 125 and 150 treatment groups, respectively). There were no participants with a mean estradiol concentration <20 μg/mL and there were 5 (21.7%) participants with a mean estradiol concentration ≥20 and <30 μg/mL (0 [0.0%], 0 [0.0%], 0 [0.0%] and 5 [83.3%] in the Cerazet, LNG VDS 75, 125 and 150 treatment groups, respectively).

In TC2 mean (SD) estradiol concentration was 46.1 (29.9) pg/mL (75.5 [41.0] pg/mL, 44.6 [26.8] pg/mL, 35.4 [3.3] pg/mL and 28.4 [8.3] pg/mL in the Cerazet, LNG VDS 75, 125 and 150 treatment groups, respectively). There were 1 (4.5%) participant with a mean estradiol concentration <20 μg/mL (0 [0.0%], 0 [0.0%], 0 [0.0%] and 1 [16.7%] in the Cerazet, LNG VDS 75, 125 and 150 treatment groups, respectively) and 6 (27.3%) participants with a mean estradiol concentration ≥20 and <30 μg/mL (1 [16.7%], 2 [50.0%], 0 [0.0%] and 3 [50.0%] in the Cerazet, LNG VDS 75, 125 and 150 treatment groups, respectively).

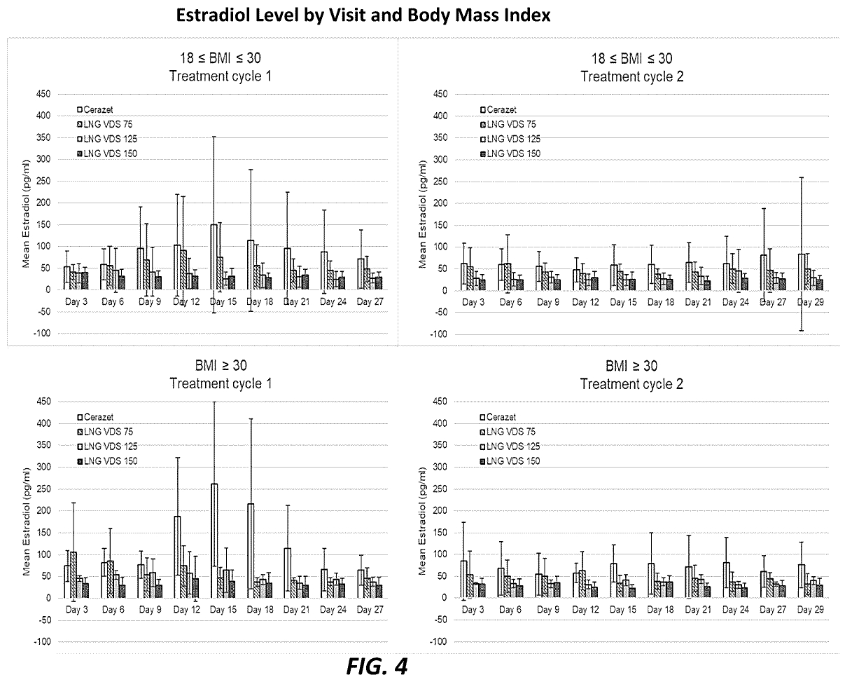

Mean estradiol levels in each treatment cycle and by BMI category for all treatment groups have been presented in FIG. 4. This figure shows how the estradiol levels decreased from TC1 to TC2 in both BMI groups although serum levels were higher in TC 1 among participants with BMI ≥30 kg/m2 compared to participants with a lower BMI.

### 4.2. Secondary Efficacy Assessment

### 4.2.1. Insler Score

Insler score results obtained by visit during pre-treatment, TC1, TC2 and post-treatment were assessed for each treatment group in the PP set and in the FAS. Insler score was assessed in 38 participants from the FAS in both TC.

In TC1 the mean (SD) maximum score obtained was 4.6 (2.1) (4.4 [1.8], 4.5 [1.8], 5.6 [2.7] and 3.5 [2.4] in the Cerazet, LNG VDS 75, 125 and 150 treatment groups, respectively). There were 9 (23.7%) participants with a maximum Insler score between 0 and 3 (3 [20.0%], 3 [27.3%], 2 [25.0%] and 1 [25.0%] in the Cerazet, LNG VDS 75, 125 and 150 treatment groups, respectively) and 20 (52.6%) with a maximum Insler score between 4 and 6 (9 [60.0%], 6 [54.5%], 2 [25.0%] and 3 [75.0%] in the Cerazet, LNG VDS 75, 125 and 150 treatment groups, respectively).

In TC2, the mean (SD) maximum Insler score obtained was 4.1 (2.1) (4.2 [1.6], 3.7 [1.8], 5.0 [3.7] and 3.4 [2.7] in the Cerazet, LNG VDS 75, 125 and 150 treatment groups, respectively). There were 13 (34.2%) with a maximum score between 0 and 3 (5 [29.4%], 4 [36.4%], 2 [40.0%] and 2 [40.0%] in the Cerazet, LNG VDS 75, 125 and 150 treatment groups, respectively) and 23 (60.5%) with a maximum score between 4 to 6 (11 [64.7%], 7 [63.6%], 2 [40.0%] and 3 [60.0%] in the Cerazet, LNG VDS 75, 125 and 150 treatment groups. respectively).

Among 12 participants with BMI≥30 kg/m2 (Table 15), the mean (SD) maximum Insler score obtained was 5.8 (1.8) during TC1 (5.4 [1.5], 4.5 [0.7], 7.3 [1.7] and 4.0 [NA] in the Cerazet, LNG VDS 75, 125 and 150 treatment groups, respectively). None of these participants had a maximum score between 0 and 3 and 7 (58.3%) participants had a maximum Insler score between 4 to 6 (3 [60.0%], 2 [100.0%], 1 [25.0%] and 1 [100.0%] in the Cerazet, LNG VDS 75, 125 and 150 treatment groups, respectively).

During TC2, the mean (SD) maximum score was 3.6 (2.0), that was assessed in only 8 participants (4.8 [0.5], 4.0 [NA], 5.0 [NA] and 0.5 [0.7] in the Cerazet, LNG VDS 75, 125 and 150 treatment groups, respectively). There were 2 (25%) participants with a Insler score between 0 and 3 (0 [0.0%], 0 [0.0%], 2 [100%] and 0 [0.0%] in the Cerazet, LNG VDS 75, 125 and 150 treatment groups, respectively) and 6 (75.0%) participants with a Insler score between 4 and 6 (4 [100%], 1 [100%], 1 [100%] and 0 [0.0%] in the Cerazet, LNG VDS 75, 125 and 150 treatment groups, respectively).

### 4.2.2. Return of Ovulation

The return of ovulation assessed in the FAS (Table 9) showed that 104 (81.9%) of 127 participants showed return of ovulation during post-treatment (23 [69.7%], 24 [80.0%], 31 [93.9%] and 26 [83.9%] in the Cerazet, LNG VDS 75, 125 and 150 treatment groups, respectively): 86 (81.9%) of 105 participants with BMI ≥18 and <30 kg/m2 (20 [74.1%], 20 [6.9%], 25 [92.6%] and 21 [84.0%] in the Cerazet, LNG VDS 75, 125 and 150 treatment groups, respectively) and 18 (81.8%) 22 participants with BMI≥30 kg/m2 (3 [50.0%], 4 [100%], 6 [100%] and 5 [83.3%] in the Cerazet, LNG VDS 75, 125 and 150 treatment groups, respectively).

TABLE 9
Return of Ovulation (Full Analysis Set)

| | LNG VDS 75 | | LNG VDS 125 | | LNG VDS 150 | | Cerazet | | All | |
|---|---|---|---|---|---|---|---|---|---|---|
| | (N = 30) | | (N = 34) | | (N = 31) | | (N = 33) | | (N = 128) | |
| Return of ovulation | | | | | | | | | | |
| Number | 30 | | 33 | | 31 | | 33 | | 127 | |
| Yes | 24 | (80.0%) | 31 | (93.9%) | 26 | (83.9%) | 23 | (69.7%) | 104 | (81.9%) |
| No | 6 | (20.0%) | 2 | (6.1%) | 5 | (16.1%) | 10 | (30.3%) | 23 | (18.1%) |
| Missing | 0 | | 1 | | 0 | | 0 | | 1 | |
| 18 ≤ BMI < 30 | | | | | | | | | | |
| Number | 26 | | 27 | | 25 | | 27 | | 105 | |
| Yes | 20 | (76.9%) | 25 | (92.6%) | 21 | (84.0%) | 20 | (74.1%) | 86 | (81.9%) |
| No | 6 | (23.1%) | 2 | (7.4%) | 4 | (16.0%) | 7 | (25.9%) | 19 | (18.1%) |
| Missing | 0 | | 0 | | 0 | | 0 | | 0 | |
| BMI ≥30 | | | | | | | | | | |
| Number | 4 | | 6 | | 6 | | 6 | | 22 | |
| Yes | 4 | (100.0%) | 6 | (100.0%) | 5 | (83.3%) | 3 | (50.0%) | 18 | (81.8%) |
| No | 0 | (0.0%) | 0 | (0.0%) | 1 | (16.7%) | 3 | (50.0%) | 4 | (18.2%) |

| | | | | | |
|---|---|---|---|---|---|
| Missing | 0 | 1 | 0 | 0 | 1 |

The same results were observed when the return of ovulation was assessed in the PP set. The mean (SD) progesterone blood levels assessed during post-treatment cycle (in the FAS) were 17.3 (15.3) pg/mL in OV+2 (12.8 [11.0], 16.4 [13.5], 15.5 [12.3] and 24.7 [20.8] in the Cerazet, LNG VDS 75, 125 and 150 treatment groups, respectively) and 29.9 (14.9) pg/mL in OV+4 (31.0 [13.5], 29.1 [17.7], 26.9 [11.7] and 33.4 [16.2] in the Cerazet, LNG VDS 75, 125 and 150 treatment groups, respectively). Similar results were obtained in the assessment of progesterone blood levels during post-treatment cycle in the PP set.

### 4.2.3. Endometrial Thickness

Endometrial thickness was assessed in order to determine any changes in the endometrial bed which were inappropriate for nidation. A thickness of <6 mm was regarded as an endometrium which was not adequate for pregnancy. It was measured as double-layer distance in longitudinal section of the uterus by TVUs at each visit.

Endometrial thickness was measured by TVU at pre-treatment, TC1, TC2 and posttreatment, on each study visit. The mean results obtained at each visit and by treatment group have been analyzed for the whole population and for the group of participants with BMI ≥30 kg/m2.

In pre-treatment, the mean (SD) endometrial thickness was 7.8 (2.1) mm (8.2 [2.9], 7.2 [1.8], 7.9 [1.3] and 7.8 [1.9] mm in the Cerazet, LNG VDS 75, 125 and 150 treatment groups, respectively).

In TC1, the mean (SD) endometrial thickness was 4.1 (1.1) (4.3 [1.3], 3.7 [0.9], 4.1 [0.8] and 4.1 [1.1] mm in the Cerazet, LNG VDS 75, 125 and 150 treatment groups, respectively).

In TC2, the mean (SD) endometrial thickness was 3.8 (1.0) mm (3.9 [1.2], 3.3 [0.9], 3.9 [0.9] and 3.9 [1.0] mm in the Cerazet, LNG VDS 75, 125 and 150 treatment groups, respectively).

In post-treatment, the mean (SD) endometrial thickness was 7.0 (1.6) mm (6.7 [2.0], 6.7 [1.6], 7.3 [1.5] and 7.4 [1.3] mm in the Cerazet, LNG VDS 75, 125 and 150 treatment groups, respectively).

In the group of participants with BMI ≥30 kg/m2, in pre-treatment, the mean (SD) endometrial thickness in this group of participants was 7.8 (2.1) mm (9.1 [1.7], 7.2 [0.9], 8.0 [1.5] and 9.1 [2.7] mm in the Cerazet, LNG VDS 75, 125 and 150 treatment groups, respectively). In TC1, the mean (SD) endometrial thickness was 4.1 (1.1) mm (5.6 [1.7], 3.6 [0.7], 4.0 [0.6] and 4.6 [1.2] mm in the Cerazet, LNG VDS 75, 125 and 150 treatment groups, respectively). In TC2, the mean (SD) endometrial thickness was 3.8 (1.0) mm (4.9 [1.1], 3.9 [1.5], 3.6 [0.6] and 3.8 [1.4] mm in the Cerazet, LNG VDS 75, 125 and 150 treatment groups, respectively). In post-treatment (Table 20), the mean (SD) endometrial thickness was 7.0 (1.6) mm (6.9 [1.5], 6.9 [0.6], 6.4 [1.8] and 6.9 [1.7] mm in the Cerazet, LNG VDS 75, 125 and 150 treatment groups, respectively).

### 4.2.4. Bleeding Patterns

Vaginal bleeding was recorded daily in the subject's diary. The results of the presence or absence of bleeding and the bleeding episode are presented for all participants.

In TC1, 1 (0.8%) participant had no bleeding during the entire cycle (in the LNG VDS 75 treatment group). The mean (SD) number of days with any bleeding was 11.2 (6.0) (10.7 [4.9], 11.5 [6.4], 11.6 [6.4] and 11.3 [6.3] days in the Cerazet, LNG VDS 75, 125 and 150 treatment groups, respectively); the mean (SD) number of days with spotting was 6.2 (4.7) (5.5 [4.4], 6.8 [5.6], 5.9 [4.0] and 6.9 [5.0] days in the Cerazet, LNG VDS 75, 125 and 150 treatment groups, respectively) and the mean (SD) number of days with slight bleeding was 3.4 (2.8) (3.2 [2.4], 2.8 [2.1], 4.6 [3.9] and 2.8 [1.8] days in the Cerazet, LNG VDS 75, 125 and 150 treatment groups, respectively).

In TC2, 22 (17.6%) participants had no bleeding (3 [9.7%], 8 [26.7%], 8 [24.2%] and 3 [9.7%] participants in the Cerazet, LNG VDS 75, 125 and 150 treatment groups). The mean (SD) days with any bleeding was 14.1 (8.9) (12.5 [6.7], 13.0 [8.2], 17.9 [9.7] and 13.2 [10.2] days in the Cerazet, LNG VDS 75, 125 and 150 treatment groups, respectively); the mean (SD) number of days with spotting was 9.2 (7.1) (7.4 [5.1], 9.3 [8.1], 10.3 [6.8] and 9.7 [8.1] days in the Cerazet, LNG VDS 75, 125 and 150 treatment groups, respectively) and the mean (SD) number of days with slight bleeding was 5.8 (4.7) (5.1 [4.0], 3.3 [2.3], 8.0 [5.2] and 6.3 [5.6] days in the Cerazet, LNG VDS 75, 125 and 150 treatment groups, respectively).

### 5. Efficacy Conclusion

A total of 128 participants were included in the FAS.

The analysis of the primary endpoint showed that all participants treated with LNG VDS showed inhibition of ovulation in both TCs. All participants treated with Cerazet also had inhibition of ovulation in both TCs, except 1 participant (with BMI ≥18 and <30 kg/m2) who ovulated during TC1.

Ovarian activity measured by Hoogland score during TC1 showed that 69% of participant had no or minimum ovarian activity, being this percentage of 84% among participants treated with LNG VDS 150; 31% presented residual ovarian activity being this percentage higher among participants treated with LNG VDS 75 (43%). Likewise, more participants with BMI ≥18 and <30 kg/m2 showed none or minimum ovarian activity (73%) compared to participants with BMI ≥30 kg/m2 (48%) although this percentage was higher if such participants were treated with LNG VDS 150 (83%). During TC2, the percentage of participants with none or minimum ovarian activity compared to TC1 was higher in the whole population (71% vs 68%), as well as among participants with BMI ≥30 kg/m2 (68% vs 48%).

The ANCOVA model showed with statistical significance that during TC2, the Hoogland score differed 1 point in participants treated with LNG VDS 150 and participants treated with Cerazet (1.8 vs 2.8, respectively) and 0.7 between those treated with LNG VDS 125 and those treated with Cerazet (2.1 vs 2.8, respectively).

The results from Landgren score confirmed that only 1 participant treated with Cerazet ovulated in TC1.

In TC1, the mean (SD) largest follicle size was 10.2 (3.8) mm, being higher in participants treated with Cerazet and LNG VDS 75 (11.7 [4.6] and 11.2 [4.2] mm, respectively) versus LNG VDS 125 and 150 (9.4 [3.4] and 8.6 [2.1] mm, respectively). the same trend was observed in TC2 with similar results.

In participants with a BMI ≥30 Kg/m2, a higher follicle size was obtained although the same differences were observed between treatment arms, however, the values obtained in TC2 were very similar to that observed for the general population.

Serum progesterone concentration were higher in TC1 in participants treated with Cerazet in the last visits of TC1 compared to those values observed in the LNG VDS treatment groups, while no important variations were observed during TC2 regardless the treatment group.

In participants with BMI ≥30 kg/m2, no high variations were observed among treatment groups in both TC1 and TC2 by study visit, although serum progesterone levels were in general lower than those observed in the general population.

Regarding the mean estradiol concentration, the value obtained in TC1 was 56.6 (54.6) pg/mL although this value was higher in the Cerazet treatment group (97.1 [84.3] pg/mL) compared to LNG VDS 75, 125 and 150 (59.3 [40.2], 37.3 [21.2] and 32.1 [12.1] pg/mL, respectively). The same trend was observed in TC2 although such values were lower compared to TC1. It is important to highlight that the percentage of participants with estradiol concentration <20 μg/mL and <30 μg/mL was noticeable higher among those treated with LNG VDS 125 and 150 compared to LNG VDS 75 and Cerazet. In participant with BMI ≥30 kg/m2, the same trend was observed although estradiol serum concentration was higher in all treatment arms in both TCs compared to the whole population.

The maximum mean Insler score obtained was 4.6 in TC1 and 4.1 in TC2 and in both TCs, participants treated with LNG VDS 150 showed a lower maximum score (3.5 in TC1 and 3.4 in TC2) compared to the other 3 treatment groups. No participant had a good condition for the sperm ascension (maximum Insler score of 10-12) in any of the two TCs and only 1 participant (treated with Cerazet) had moderate condition for the sperm ascension (maximum Insler score of 7-9) in TC2. Among participants with BMI ≥30 kg/m2 the maximum Insler score in TC1 was higher compared to the whole population (5.8) and no participants had a mean score between 0 and 3. Among these, no participants had maximum Insler score of 10-12 in any TC and maximum Insler score of 7-9 was only reached by 3 and 2 participants treated with LNG VDS 125 and Cerazet, respectively, only in TC1.

In post-treatment, the ovulation was recovered in more than 80% of participants (80.8%, 93.9% and 83.9% in the LNG VDS 75, 125 and 150 treatment groups vs 69.7% in the Cerazet treatment group).

### Pharmacokinetic Evaluation

Pharmacokinetic parameters of LNG and SHBG levels were determined in a subgroup of 55 participants using LNG VDS; 41 of them had a BMI between 18≤ and <30 kg/m2 (15, 14 and 12 participants for the LNG VDS 75, 125 and 150 treatment groups, respectively) and 14 of them had a BMI≥30 kg/m2 (3, 7 and 4 participants for the LNG VDS 75, 125 and 150 treatment groups, respectively).

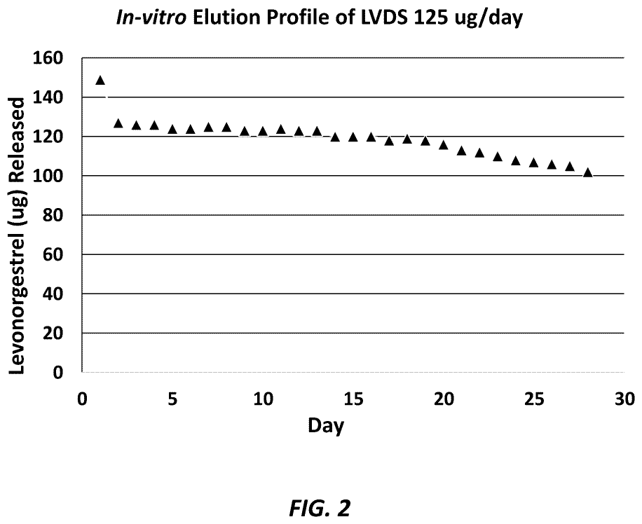

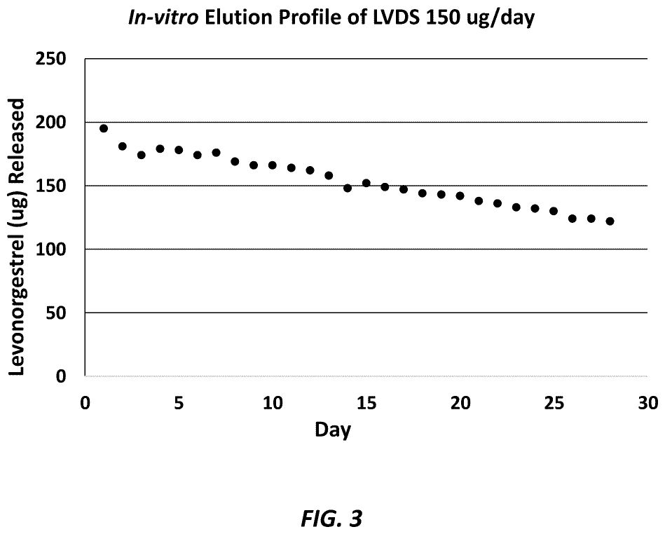

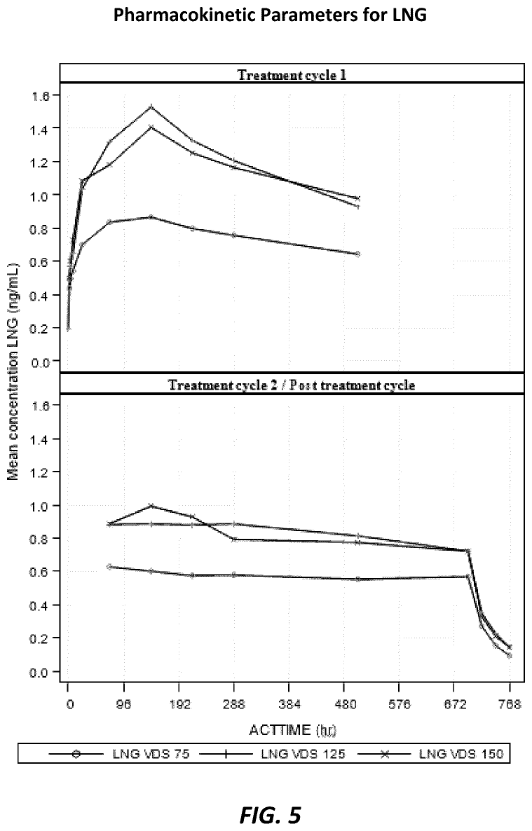

FIGS. 2 and 3 show the mean concentration-time profiles of LNG and SHBG, respectively, of participants from the LNG VDS 75, 125 and 150 treatment groups; and FIGS. 4 and 5 show the same parameters (mean concentration-time profiles of LNG and SHBG) categorized by BMI.

### 5.1. LNG Pharmacokinetics of LNG VDS 75, 125 and 150 Treatment Groups

Following insertion of the LNG VDS at dose levels of 75, 125 and 150 μg/day, plasma concentrations of LNG were evident from first sampling (2 h post insertion) in all subjects, in all cycles at all dose levels.

In TC1, maximal concentrations overall were observed at 116, 123 and 113 hours following dosing at 75, 125 and 150 μg/day, respectively. In the BMI category ≥18 and <30 kg/m2, the Tmax values observed were 115, 110 and 98.667 hours, following insertion at 75, 125 and 150 μg/day, respectively, and in participants with BMI ≥30 kg/m2 the Tmax values observed were 120, 151 and 156 hours following dosing at 75, 125 and 150 μg/day, respectively.

Following the peak plasma concentrations of LNG at Tmax, plasma levels remained elevated with minimal peak to trough fluctuation representative of prolonged release of LNG over TC1 in all dose levels and in all BMI categories.

In TC1, maximal plasma concentrations observed overall were 0.950, 1.548 and 1.426 ng/mL following dosing at 75, 125 and 150 μg/day. In participants with BMI ≥18 and <30 kg/m2 the Cmax values were 1.011, 1.829 and 1.539 ng/mL, and in participants with BMI ≥30 kg/m2 the Cmax values were 0.647, 0.987 and 1.090 following dosing at 75, 125 and 150 μg/day, respectively.

Exposure as assessed by AUC(0-t) observed overall in TC1 were 340, 571 and 468 h*ng/mL following dosing at 75, 125 and 150 μg/day. In the BMI ≥18 and <30 kg/m2 category the AUC(0-t) values were 357, 662 and 490 h*ng/mL, and in participants with BMI ≥30 kg/m2 the AUC(0-t) values were 253, 389 and 403 following dosing at 75, 125 and 150 μg/day, respectively.

The clearance values measured following the extravascular dosing of LNG VDS at 75, 125 and 150 μg/day in TC1 were 76.383, 96.103 and 106 mL/h, and in participants with BMI ≥18 and <30 kg/m2 the values were 66.816, 84.964 and 101 mL/h, and where BMI was ≥30 kg/m2 the values determined were 119, 125 and 123 mL/h.

The volume of distribution measured following the extravascular dosing of LNG VDS at 75, 125 and 150 μg/day the values in TC1 were 101, 96.294, 103 L, and in participants with BMI ≥18 and <30 kg/m2 the values were 99.117, 85.227 and 94.116 L and where BMI was ≥30 kg/m2 the values determined were 111.991, 124.941 and 140.244 L.

In TC2, maximal concentrations of LNG overall were observed at 283, 192 and 192 hours following dosing at 75, 125 and 150 μg/day, respectively. In participants with BMI ≥18 and <30 kg/m2, the Tmax values observed were 269, 156 and 199 hours, following insertion at 75, 125 and 150 μg/day, respectively. In participants with BMI ≥30 kg/m2, the Tmax values observed were 352, 276 and 174 hours, following dosing at 75, 125 and 150 μg/day, respectively.

Following the peak plasma concentrations of LNG at Tmax, plasma levels remained elevated with minimal peak to trough fluctuation representative of prolonged release of LNG over TC2 in all dose levels and in all BMI categories.

In TC2, maximal plasma concentrations observed overall were 0.654, 0.987 and 0.925 ng/mL following dosing at 75, 125 and 150 μg/day. In participants with BMI ≥18 and <30 kg/m2 the Cmax values were 0.695, 1.085 and 0.995 ng/mL, and in participants with BMI ≥30 kg/m2 the Cmax values were 0.449, 0.757 and 0.842 ng/mL following dosing at 75, 125 and 150 μg/day, respectively.

In TC2, exposure as assessed by AUC(0-t) observed overall were 363, 538 and 505 h*ng/mL following dosing at 75, 125 and 150 μg/day. In participants with BMI ≥18 and <30 kg/m2 the AUC(0-t) values were 382, 584 and 517 h*ng/mL, and in participants with BMI ≥30 kg/m2 the AUC(0-t) values were 269, 432, 473 h*ng/mL following dosing at 75, 125 and 150 μg/day, respectively.

In TC2, the clearance values measured following the extravascular dosing of LNG VDS at 75, 125 and 150 μg/day were 25.705, 75.382 and 62.028 mL/h, and in participants with BMI ≥18 and <30 kg/m2 the values were 25.449, 57.623 and 52.200 mL/h, and where BMI was ≥30 kg/m2 the values determined were 27.240, 116.821 and 91.512 mL/h.

In TC2, the volume of distribution measured following the extravascular dosing of LNG VDS at 75, 125 and 150 μg/day the values were 138, 140 and 172 L, and in participants with BMI ≥18 and <30 kg/m2 the values were 131, 118 and 167 L and in participants with BMI ≥30 kg/m2 the values determined were 177, 1912 and 187 L.

In both TC1 and TC2, whilst terminal elimination half-life values are reported, these do not represent the elimination kinetics of LNG, as over the sampling period a constant input of LNG is delivered from the VDS, therefore these values are not interpreted or discussed.

In summary, in TC1 overall an increase in exposure (as determined by Cmax and AUC(0-t)) was observed over the dosing range, and participants with BMI ≥30 kg/m2 showed lower exposure, lower in participants with BMI ≥18 and <30 kg/m2.

In TC2 overall an increase in exposure (as determined by Cmax and AUC(0-t)) was observed between 75 and 125 μg/day dose levels; however, no clear overall difference occurred between 125 and 150 μg/day dose levels. In participants with BMI ≥30 kg/m2 these tended to demonstrate a lower exposure (as determined by Cmax and AUC(0-t)), a lower clearance and a larger volume of distribution than observed in participants with BMI ≥18 and <30 kg/m2. As observed overall and in both BMI categories no clear difference in exposure was observed between the 125 and 150 μg/day dose levels. The plasma clearance observed in TC2 appeared different over the dose levels, with a lower clearance observed for both BMI categories at 75 μg/day when compared to dosing at 125 and 150 μg/day.

### Example 3

A Multi-center, phase 2, dose finding, double-blind, randomized clinical trial to assess the efficacy and safety of levonorgestrel (LNG) vaginal delivery system (VDS), releasing during 28 days in continuous regimen of 75 mcg/day and 125 mcg/day, for the management of moderate to severe pain associated with endometriosis versus placebo after 3 medication cycles.

### Study Design:

Multi-center clinical trial in female subjects ≥18 and <45 years of age with surgically confirmed diagnosis of endometriosis and with moderate to severe pain during the most recent menses and with non-menstrual pelvic pain (NMPP) in the prior month. The clinical trial consists of a screening period (up to 100 days), a baseline cycle and a treatment period consisting of 3 placebo-controlled, double-blind medication cycles.

The subjects will be randomized to receive either LVDS 75 mcg/day, LVDS 125 mcg/day or placebo vaginal ring. The vaginal rings will be inserted on the first day of the next menstrual bleeding after Visit 1b and should be change every 28 days. If the menstrual bleeding starts in the evening, and the subject prefers to insert the ring in the morning, then she may insert the next day [Day 2 of the menstrual bleeding]. Afterwards, the subjects will come to the site at Visit 2 and Visit 3 on Day 20 (+6) of the 1st and 3rd medication cycle. The end-of treatment visit (Visit 4/early discontinuation visit [EDV]) will be performed up to 3 days after the last day using the ring.

### Study Objectives:

### Primary Objective:

To demonstrate the efficacy of levonorgestrel vaginal delivery system (LVDS) of two doses LVDS 75 mcg/day and LVDS 125 mcg/day compared to placebo for the management of Dysmenorrhea and non-menstrual pelvic pain in women with endometriosis associated pain

### Primary Endpoint

Proportion of patients who meet the dysmenorrhea responder criteria after 12 weeks of treatment with LVDS 75 mcg and LVDS 125 mcg compared to placebo, achieving a mean reduction in dysmenorrhea NRS scores of at least 2.8 points and no increase in use of analgesic medications as recorded in a daily electronic diary compared with the Baseline pain assessment period.
Proportion of patients who met the non-menstrual pelvic pain (NMPP) responder criteria after 12 weeks of treatment with LVDS 75 mcg and LVDS 125 mcg compared to placebo, achieving a mean reduction in non-menstrual pelvic pain NRS scores of at least 2.1 points and no increase in use of analgesic medications as recorded in a daily electronic diary compared with the Baseline pain assessment period

### Secondary Objectives:

To determine the benefit on function measured by the Endometriosis Health Profile-30 pain domain.
To determine the benefit on function measured by the Endometriosis-associated quality of life Health Profile-30 non-pain domain.
To determine the benefit on dysmenorrhea, pelvic pain and overall pain in NRS
To determine the benefit on the Patient Global Assessment for dysmenorrhoea, NMPP, overall pain severity and functional impairment
To determine change in rescue medication use

### Secondary Endpoints:

Change from baseline to week 12 in the Endometriosis Health Profile-30 pain domain score
Change from baseline to week 12 on endometriosis-associated quality of life, measured by non-pain domains of the EHP-30
Change from baseline after 1 month, 2 month and end of treatment (after 12 weeks) on the mean dysmenorrhea, pelvic pain and overall pain in NRS scores
Changes from baseline after 1 month, 2 month and end of treatment (after 12 weeks) in Patient Global Assessment (PGA) for dysmenorrhea, NMPP, pain, and function
Changes in rescue medication use (per 24 hours) after 1 and 3 medication cycle(s) compared to baseline

### Dose and Route of Administration:

### Test Product

Levonorgestrel Vaginal Delivery System (LVDS) 75 mcg/day.

Levonorgestrel Vaginal Delivery System (LVDS) 125 mcg/day.

### Reference Product

Placebo vaginal ring will be manufactured to match the shape, size and color of the LVDS

Each LVDS contains approximately 10 mg LNG, which is designed to release 75 mcg/day and 125 mcg/day.

### Duration of Treatment

Three treatment cycles of 28 days of duration.

The subjects will insert the vaginal ring the first day of menses and use continuously for 28 days. After 28 days the subjects will exchange the vaginal ring (take out the current one and insert the next vaginal ring without free time interval).

The investigator will instruct the subject the ring should not be removed for more than 3 hours within 24 hours.

### Statistical Methods

The efficacy analyses will be conducted using a modified Intent-to-Treat (mITT) population defined as all randomized patients who have had at least one dose of randomized study drug, unless otherwise specified in Statistical Analysis Plan. The randomization ratio will be 1:1:1 among the 3 treatment arms:
LVDS 75 mcg/day. (N≈50)
LVDS 125 mcg/day. (N≈50)
Placebo (N≈50)

This study has two primary endpoints defined as:
Proportion of patients who meet the dysmenorrhea responder criteria after 12 weeks of treatment with 75 mcg/day and LVDS 125 mcg/day compared to placebo.
Proportion of patients who met the non-menstrual pelvic pain (NMPP) responder criteria after 12 weeks of treatment with 75 mcg/day and LVDS 125 mcg/day compared to placebo.

Baseline pain assessment will be based on the average of the values observed during baseline cycle up to the day prior to the date of first dose of randomized study drug.

A responder (defined for dysmenorrhea and NMPP separately) is defined as a patient who did not have an increase in the use of rescue analgesic medications during the Week 12/EOT pain assessment period (last 35 days prior to last dose of study drug) compared with the Baseline pain assessment period and whose reduction in pain exceeds the defined response threshold as follows:
For dysmenorrhoea achieving a mean reduction NRS scores of at least 2.8 points
For nonmenstrual pelvic pain achieving a mean reduction NRS scores of at least 2.1 points

Patients who complete <5 weeks of treatment will be considered non-responders for both dysmenorrhea and non-menstrual pelvic pain. For patients who complete at least 5 weeks of treatment, responder status for dysmenorrhea and non-menstrual pelvic pain will be defined as follows:
For dysmenorrhea, the responder status will be defined using the following rules taking analgesic use into consideration and requiring at least 2 days of dysmenorrhea NRS scores in the electronic diary, then the average will be calculated and use in the assessment of the dysmenorrhea responder status. Requiring a minimum of 2 days of dysmenorrhea NRS scores is considered reasonable since a typical menstrual cycle has bleeding days ranging from 3 to 7 days.
For non-menstrual pelvic pain, the responder status was defined using the following rules, taking analgesic use into consideration, and requiring at least 14 days of scores for non-menstrual pelvic pain reported in the electronic diary, then the average non-menstrual pelvic pain score will be used in the assessment of the non-menstrual pelvic pain responder status. Requiring minimum 14 days of non-menstrual pelvic pain scores is considered reasonable since this corresponds to at least half of the non-menstrual days from a typical 28-day menstrual cycle.

The responder rate for placebo arm is assumed to be between 30 to 35%.

1. A method for providing contraception in a female subject, comprising continuously administering levonorgestrel to said subject, wherein the levonorgestrel is continuously administered in an amount ranging from about 60 μg/day to about 100 μg/day, wherein the administration route is vaginal administration.

2. A method for treating endometriosis, endometriosis associated pelvic pain (EAPP) and/or dysmenorrhea in a female subject, comprising continuously administering levonorgestrel to said subject, wherein the levonorgestrel is continuously administered in an amount ranging from about 60 μg/day to about 160 μg/day, and wherein the administration route is vaginal administration.

3. The method of claim 2, wherein said treatment also provides contraception.

4. The method of claim 1, wherein no further contraceptive ingredient is administered to the female subject at the same time.

5. The method of claim 1, wherein said administering of Levonorgestrel also induces amenorrhea.

6. A drug delivery device comprising:
(a) a core comprising a polymer;
(b) a sheath substantially or completely surrounding said core,
said sheath comprising a polymer; and
(c) Levonorgestrel dissolved or dispersed in said core and/or said sheath, wherein the total amount of Levonorgestrel present in said core and/or said sheath is between about 9 mg to about 11 mg.

7. The drug delivery device of claim 6, wherein said device has a drug release profile wherein the device releases:
(i) no more than about 150 μg of levonorgestrel during an initial 24-hour period of release; and
(ii) about 60 μg to 90 μg of levonorgestrel per day for at least 27 days after the initial 24 hours period of release;
when the device is subjected to an in vitro release test in a 0.2M sodium acetate buffer with 1.0% sodium lauryl sulfate (SLS) surfactant adjusted to pH 4.2 medium.

8. The drug delivery device of claim 6, wherein said device has a drug release profile wherein the device releases
(i) no more than 250 μg of levonorgestrel during an initial 24-hour period of release; and
(ii) about 90 μg to 150 μg of levonorgestrel per day for at least 27 days after the initial 24 hours period of release;
when the device is subjected to an in vitro release test in a 0.2M sodium acetate buffer with 1.0% sodium lauryl sulfate (SLS) surfactant adjusted to pH 4.2 medium.

9. The drug delivery device of claim 6, wherein said device has a drug release profile wherein the device releases
(i) no more than about 300 μg of levonorgestrel during an initial 24-hour period of release; and
(ii) about 110 μg to 180 μg of levonorgestrel per day for at least 27 days after the initial 24-hour period of release;
when the device is subjected to an in vitro release test in a 0.2M sodium acetate buffer with 1.0% sodium lauryl sulfate (SLS) surfactant adjusted to pH 4.2 medium.

10. The drug delivery device of claim 6, wherein said device provides a mean Cmax value for levonorgestrel of less than 1 ng/ml after one treatment cycle of 28 days and of less than 0.7 ng/ml after two treatment cycles of 28 days each and a mean AUC (0.-t) value of less than 350 h*ng/ml after one treatment cycle of 28 days and of less than 370 h*ng/ml after two treatment cycles of 28 days each, in a female subject, after the delivery device was placed intravaginally within the subject's body.

11. The drug delivery device of claim 6, wherein said device provides a mean Cmax value for levonorgestrel of less than 1.6 ng/ml after one treatment cycle of 28 days and of less than 1 ng/ml after two treatment cycles of 28 days each and a mean AUC (0-t) value of less than 580 h*ng/ml after one treatment cycle of 28 days and of less than 540 h*ng/ml after two treatment cycles of 28 days each in a female subject, after the delivery device was placed intravaginally within the subject's body.

12. The drug delivery device of claim 6, wherein the device does not comprise any further contraceptive ingredient.

13. The drug delivery device according to claim 6, wherein
(i) levonorgestrel is present in the core at a concentration of about 0.20 to about 1.00 wt % based on the total core weight; and/or
(ii) the sheath has a thickness comprised from about 5 to about 500 μm, for example from about 50 to about 200 μm.

14. The drug delivery device according to claim 6, wherein the drug delivery device has a shape selected from a spiral shape, a T-shape or a ring shape, for example wherein the drug delivery device has a ring shape, or wherein the device is a vaginal ring.

15. (canceled)

16. The method according to claim 4, wherein the contraceptive ingredient is estrogen.

17. The drug delivery device according to claim 6, wherein the core comprising a polymer comprises polyurethane.

18. The drug delivery device according to claim 6, wherein the sheath comprising a polymer comprises ethylene vinyl acetate copolymer with a vinyl acetate content from about 10 to 40% w/w.

19. The drug delivery device according to claim 12, wherein the contraceptive ingredient is estrogen.

20. The drug delivery device according to claim 13, wherein the sheath has a thickness comprised from about 50 to about 200 μm.

21. The drug delivery device according to claim 14, wherein the drug delivery device has a ring shape.

22. The drug delivery device according to claim 14, wherein the device is a vaginal ring.

23. The method of claim 2, wherein no further contraceptive ingredient is administered to the female subject at the same time.

24. The method according to claim 23, wherein the contraceptive ingredient is estrogen.

25. The method of claim 2, wherein said administering of Levonorgestrel also induces amenorrhea.
